(19) United States
(12) Patent Application Publication
(10) Pub. No.: US 2024/0350606 A1
(43) Pub. Date: Oct. 24, 2024

(54) VACCINE COMPOSITIONS AND METHODS FOR ENHANCED ANTIGEN-SPECIFIC VACCINATION

(71) Applicants: DUKE UNIVERSITY, DURHAM, NC (US); THE JOHNS HOPKINS UNIVERSITY, BALTIMORE, MD (US)

(72) Inventors: YOU-WEN HE, DURHAM, NC (US); HEQIANG SUN, DURHAM, NC (US); THOMAS AUGUST, BALTIMORE, MD (US); JUN LIU, BALTIMORE, MD (US); YADONG WEI, BALTIMORE, MD (US)

(73) Assignees: DUKE UNIVERSITY, DURHAM, NC (US); THE JOHNS HOPKINS UNIVERSITY, BALTIMORE, MD (US)

(21) Appl. No.: 18/665,046

(22) Filed: May 15, 2024

Related U.S. Application Data
(63) Continuation of application No. 17/052,030, filed on Oct. 30, 2020.
(60) Provisional application No. 62/666,355, filed on May 3, 2018.

(51) Int. Cl. A61K 39/00; A61K 39/245; A61P 35/00; C07K 14/005; C07K 14/47; C12N 15/85

(52) U.S. Cl. CPC A61K 39/001192; A61K 39/0008; A61K 39/001104; A61K 39/001106; A61K 39/001109; A61K 39/001184; A61K 39/001186; A61K 39/245; A61P 35/00; C07K 14/005; C07K 14/4738; C07K 14/4748; C12N 15/85; A61K 2039/5154; A61K 2039/53; A61K 2039/585; C07K 2319/01; C07K 2319/02; C07K 2319/06; C07K 2319/40; C12N 2710/16134; C12N 2800/22; C12N 2840/203

(57) ABSTRACT

Vaccine design, polycistronic vaccine constructs, compositions, and methods comprising nucleic acids (DNA, RNA), peptides, proteins and derivatives thereof, including cells and cell-lines, for enhanced antigen-specific vaccination.

### CROSS REFERENCED APPLICATIONS

This application is a divisional of U.S. patent application Ser. No. 17/052,030, filed Oct. 30, 2020, which is a U.S. National Phase Entry of International Patent Application No. PCT/US2019/030643, filed May 3, 2019, which claims the benefit of U.S. Provisional Application No. 62/666,355, filed on May 3, 2018, which is incorporated herein by reference in its entirety.

### SEQUENCE LISTING

This application contains a sequence listing that has been submitted electronically in XML format and is hereby incorporated by reference in its entirety. The XML copy, created on May 14, 2024, is named D1181200WO_Sequence_Listing and is 22,165 bytes in size.

### TECHNICAL FIELD OF THE INVENTION

The invention relates generally to vaccine design, polycistronic vaccine constructs, vaccine compositions, and methods of use thereof, designed for enhanced antigen-specific vaccination. The aforementioned polycistronic vaccine constructs, vaccine compositions and methods also concern related cells and cell-lines for replicating or expressing the nucleic acid constructs, or for vaccine delivery.

### BACKGROUND OF THE INVENTION

Nucleic acid vaccines are an emerging alternative for the prevention and treatment of infectious diseases as well as for pathologies such as cancer, allergies, autoimmune diseases, and drug dependencies. These vaccines induce the expression of encoded antigenic/therapeutic proteins or peptides (e.g., derived from a pathogen, a human self-protein, or a malignant neoplasm) in the body of an immunized (vaccinated) subject, and elicit an adaptive immune response, including humoral and cellular immune responses, as well as activate innate immune responses.

Nucleic acid vaccines offer distinct advantages over conventional vaccines in terms of safety, ease of fabrication, and stability. However, a general challenge with nucleic acid vaccines is their poor immunogenicity and therefore their lack of potency and clinical efficacy. Thus, there is a need to develop nucleic acid vaccines designed with improved immunogenicity and methods of use, thereof to provide potent antigen-specific immunization.

The immunogenicity of other current forms of vaccines, including attenuated pathogens, protein and peptide vaccines needs further improvement. For example, the protection rate of current Hepatitis B vaccine (HBV) vaccine is ˜80% in healthy populations, and with current influenza vaccines, the efficacy has been reported to range from 10% to 60%.

As discussed herein, aspects of the present invention address the aforementioned challenges and unmet needs by providing, inter alia, polycistronic vaccine constructs (DNA, RNA, protein, peptide), nucleic acid vaccine compositions/formulations, peptide or protein vaccine compositions, and methods of use thereof, for concurrently eliciting an enhanced activation of each of the three arms of the adaptive immune response: CD8⁺ cytolytic T lymphocyte (CTL), CD4⁺ helper T lymphocyte (HTL), and antibody. In particular, the polycistronic vaccine constructs provided by the invention express at least one target antigen and comprise a plurality of independent cistrons operably linked to a single promoter, wherein each independent cistron encodes a modified target antigen comprising an in-frame fusion protein of the target antigen and at least one specific domain that dictates the processing and presentation of the antigen. In certain embodiments, the domain comprises a destabilization domain (D.D.), a lysosome-associated membrane protein (LAMP) domain and a signal sequence (s.s.). Further, the present invention provides DNA and RNA constructs and methods of use thereof, to enhance induced by dendritic cell (D.C.) vaccines and other cellular vaccines, for example, peripheral blood mononuclear cells (PBMCs), erythrocytes, B lymphocytes, gammadelta T lymphocytes, monocytes, and Langerhans cells as cellular carriers of specific antigens.

### BRIEF SUMMARY OF THE INVENTION

The present invention provides polycistronic vaccine constructs for expressing at least one target antigen, the construct comprising a plurality of independent cistrons operably linked to a single promoter, wherein each independent cistron encodes a modified target antigen comprising an in-frame fusion protein of the target antigen and at least one specific domain selected from a destabilization domain (D.D.), a lysosome-associated membrane protein (LAMP) domain and a signal sequence (s.s.). In certain embodiments, the polycistronic vaccine constructs further comprise nucleotide sequences corresponding to a 5′ untranslated region (5′ UTR), a 3′ untranslated region (3′ UTR) which includes a poly A tail, and optionally a terminal immunoenhancing (IE) sequence comprising two complementary single-stranded RNA sequences separated by a small loop sequence. In certain embodiments, the IE sequence comprises a 3′-terminal double-stranded RNA spanning 50-5000 base-pairs. In particular embodiments, the double-stranded RNA comprises polyG:C or polyA:U. In certain embodiments, the double-stranded RNA is a random sequence comprising a combination of A, U, G, and C, wherein the random sequence is optimized to have no or little sequence similarity with any endogenous mammalian RNA sequences. In certain embodiments, the promoter is a mammalian promoter, a viral promoter, a T3 promoter, a T7 promoter, or an SP6 promoter. In certain embodiments, in any of the polycistronic vaccine constructs set forth herein, the target antigen is derived from a pathogen, a human self-protein, a tumor antigen, or any combination thereof. In particular embodiments, the tumor antigen comprises a tumor specific antigen, a tumor associated antigen or a neoantigen. In certain embodiments, the tumor antigen is selected from the group consisting of any of the tumor antigens set forth herein. In particular embodiments, the tumor antigen comprises a tumor associated antigen comprising human gp100. In certain embodiments, the target antigen comprises a viral pathogen. In particular embodiments, the viral pathogen is selected from the group consisting of influenza virus, human papillomavirus (HPV), hepatitis B virus (HBV), hepatitis C virus (HCV), Epstein-Barr virus (EBV), dengue virus, and human immunodeficiency virus (HIV). In particular embodiments, in any of the polycistronic vaccine constructs set forth herein, the independent cistrons are operably linked by one or more internal ribosomal entry sites (IRES) or an in-frame 2A self-cleaving peptide-based cleavage site. In particular embodiments, the IRES comprises a nucleic acid sequence derived from an encephalomyocarditis virus. In certain embodiments, in any of the polycistronic vaccine constructs set forth herein, the at least one specific domain is fused to the target antigen at the N-terminus, the C-terminus, or both the N-terminus and the C-terminus.

In certain embodiments, any of the polycistronic vaccine constructs set forth herein comprise at least two independent cistrons. In particular embodiments, one of the independent cistrons encodes a modified target antigen comprising a D.D. domain and a second independent cistron encodes a modified target antigen comprising a LAMP domain. In particular embodiments, one of the independent cistrons encodes a modified target antigen comprising a D.D. domain and a second independent cistron encodes a modified target antigen comprising an s.s. domain. In certain embodiments, the D.D. domain comprises a wild type human protein, a mutant human protein, a bacterial protein, a viral protein, or any variant/derivative thereof that undergoes proteasome-mediated degradation. In certain embodiments, the D.D. domain comprises a destabilizing sequence that is identified from a screening assay from a pool of mutants of an endogenous protein. In particular embodiments, the destabilizing mutant is selected from the group consisting of human FKBP12, F15S, V24A, L30P, E60G, M66T, R71G, D100N, E102G, K105I, E107G, L106P, and any mutations or combinations thereof. In particular embodiments, the D.D. domain comprises cyclin A, cyclin C, cyclin D, or cyclin E. In particular embodiments, the D.D. domain comprises IκB, wherein the IκB undergoes phosphorylation-dependent polyubiquitination and proteasome-mediated degradation upon activation by a surface signal. In certain embodiments, the proteasome-mediated degradation is ligand-induced. In certain embodiments, the human protein (which comprises a D.D. domain) is a known receptor for a small molecule ligand and wherein the ligand is conjugated to a compound that interacts with a E3 ubiquitin ligase or an adaptor protein to induce proteasome-mediated degradation. In particular embodiments, the adaptor protein is cereblon and the compound to be conjugated to the ligand is thalidomide, pomalidomide, lenalidomide, or a structurally related compound. In particular embodiments, the E3 ubiquitin ligase is VHL and the compound to be conjugated to the ligand is a VHL-binding small molecule. In certain embodiments, any of the polycistronic vaccine constructs set forth herein comprise three independent cistrons. In particular embodiments, a first independent cistron encodes a modified target antigen comprising a LAMP domain, a second independent cistron encodes a modified target antigen comprising a D.D. domain, and a third independent cistron encodes a modified target antigen comprising an s.s. domain.

In certain embodiments, the present invention provides a vaccine composition comprising any of the polycistronic vaccine constructs set forth herein. In particular embodiments, the vaccine composition comprises a DNA vaccine. In particular embodiments, the vaccine composition comprises an RNA vaccine. In certain embodiments, the RNA vaccine is produced by in vitro transcription of the DNA construct followed by 5′-capping of the RNA. In certain embodiments, the RNA comprises chemically modified nucleotide building blocks to enhance stability and cellular uptake in vivo. In certain embodiments, any of the vaccine compositions set forth herein comprise formulation of the DNA or RNA into nanoparticles for delivery.

In certain embodiments, the present invention provides a method for modulating an immune response in a subject comprising administering any of the polycistronic vaccine constructs or the vaccine compositions set forth herein. In certain embodiments, the present invention provides a method for providing enhanced antigen-specific vaccination in a subject comprising administering any of the polycistronic vaccine constructs or the vaccine compositions set forth herein. In certain embodiments, the present invention provides a method for inducing a therapeutic immune response against a target antigen derived from a pathogen, a human self-protein or a malignant neoplasm comprising administering any of the polycistronic vaccine constructs or the vaccine compositions set forth herein. In particular embodiments of any of the methods provided by the present invention, the method comprises an increase in CD8+ cytolytic T lymphocytes (CTL), CD4+ helper T lymphocytes (HTL), antibodies, or a combination thereof. In particular embodiments of any of the foregoing methods, the method comprises an increase in production of one or more cytokines selected from the group consisting of Interleukin-2 (IL-2), Perforin, Granzyme B, Interferon gamma (IFN-γ), tumor necrosis factor alpha (TNF-α), Interleukin-4 (IL-4), Interleukin-5 (IL-5), Interleukin-6 (IL-6) and Interleukin-10 (IL-10).

In certain embodiments, the present invention provides a nucleic acid vector for expressing a target antigen for eliciting an enhanced antigen-specific T cell response, the vector encoding a fusion polypeptide comprising the target antigen and a destabilization domain (D.D.). In certain embodiments, the fusion polypeptide (encoded by the nucleic acid vector) further comprises a LAMP domain. In certain embodiments, the target antigen (encoded by the nucleic acid vector) is derived from a pathogen, a human self-protein, or a malignant neoplasm. In particular embodiments, the target antigen is cytomegalovirus (CMV) pp65.

In certain embodiments, the present invention provides a method of manufacturing mRNA-loaded dendritic cells, the method comprising the steps of: (a) providing dendritic cells; and (b) transfecting the immature dendritic cells with one or more messenger RNA (mRNA) species transcribed in vitro from any of the polycistronic vaccine constructs set forth herein or from the nucleic acid vectors set forth herein. In certain embodiments of the method, the dendritic cells are provided by transdifferentiating autologous peripheral blood mononuclear cells into immature dendritic cells. In particular embodiments, the method comprises culturing the immature dendritic cells to obtain mature dendritic cells (mDC).

In certain embodiments, the present invention provides an isolated dendritic cell comprising one or more messenger RNA (mRNA) species transcribed in vitro from any of the polycistronic vaccine constructs set forth herein or from the nucleic acid vectors set forth herein. In certain embodiments, the present invention provides a dendritic cell vaccine composition comprising the isolated dendritic cell set forth herein. In certain embodiments, the present invention provides a therapeutic composition comprising the isolated dendritic cell set forth herein.

In certain embodiments, the present invention provides a dendritic cell vaccine composition comprising a first isolated dendritic cell and a second isolated dendritic cell, wherein the first dendritic cell and the second dendritic cell each comprise one or more messenger RNA (mRNA) species transcribed in vitro from any of the polycistronic vaccine constructs set forth herein or from the nucleic acid vectors set forth herein. In particular embodiments, the mRNA species or nucleic acid vector of the first isolated dendritic cell is different from the mRNA species or nucleic acid vector of the second isolated dendritic cell. In certain embodiments, the present invention provides a therapeutic composition comprising the first isolated dendritic cell and the second isolated dendritic cell set forth herein.

In certain embodiments, the present invention provides a method for enhancing vaccine-induced T-lymphocyte responses comprising administering to a subject in need thereof, any of the dendritic cell vaccines set forth herein, or the therapeutic compositions set forth herein. In particular embodiments of the method, the method the T-lymphocyte response comprises an increase in CD8⁺ cytolytic T lymphocytes (CTL), CD4⁺ helper T lymphocytes (HTL), or a combination thereof.

In certain embodiments, the present invention provides a method for eliciting an immune response to a cancer cell that expresses a tumor antigen, comprising administering to a subject in need thereof an effective amount of any of the dendritic cell vaccines set forth herein, or the therapeutic compositions set forth herein, wherein the effective amount of the composition is sufficient to elicit the immune response to the cancer cell that expresses the tumor antigen. In certain embodiments of the method, the subject is suffering from a tumor selected from the group consisting of glioblastoma, bladder cancer, breast cancer, ovarian cancer, pancreatic cancer, and gastric cancer, cervical cancer, colon cancer, endometrial cancer, head and neck cancer, lung cancer, melanoma, multiple myeloma, leukemia, non-Hodgkin's lymphoma, prostate cancer, rectal cancer, malignant melanoma, alimentary/gastrointestinal tract cancer, liver cancer, skin cancer, lymphoma, kidney cancer, muscle cancer, bone cancer, brain cancer, eye or ocular cancer, rectal cancer, colon cancer, cervical cancer, bladder cancer, oral cancer, benign and malignant tumors, stomach cancer, corpus uteri, testicular cancer, renal cancer, throat cancer, acute lymphocytic leukemia, acute myelogenous leukemia, Ewing's Sarcoma, Kaposi's Sarcoma, basal cell carcinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, neurofibromatosis, tuberous sclerosis, hemangiomas, and lymphangiogenesis. In certain embodiments of the method, the immune response comprises an increase in CD8⁺ cytolytic T lymphocytes (CTL), CD4⁺ helper T lymphocytes (HTL), or a combination thereof.

In certain embodiments, the present invention provides a method for eliciting an immune response to a viral antigen comprising administering to a subject in need thereof an effective amount of any of the dendritic cell vaccines set forth herein, or the therapeutic compositions set forth herein, wherein the effective amount of the composition is sufficient to provide vaccination against the viral antigen. In particular embodiments of the method, the viral antigen is selected from the group consisting of influenza virus, human papillomavirus (HPV), hepatitis B virus (HBV), hepatitis C virus (HCV), Epstein-Barr virus (EBV), dengue virus, and human immunodeficiency virus (HIV).

In certain embodiments, the present invention provides a method for delivering any of the vaccine compositions set forth herein comprising co-administering as a mixture two or more DNA constructs, RNA constructs, or any combination thereof. In particular embodiments, the method comprises co-administering an immunoadjuvant selected from the group consisting of polyIC, polyICLC, CpG, and other TLR ligands to activate dendritic cells.

In certain embodiments, the present invention provides a method for enhancing immune response and vaccination efficacy, the method comprising administering to a subject in need thereof a composition comprising any of the isolated dendritic cells, or the vaccine compositions, the dendritic cell vaccines, or the therapeutic compositions set forth herein. In particular embodiments, the method comprises co-administering an adjuvant that activates dendritic cells. In particular embodiments, the adjuvant is selected from the group consisting of polyIC, polyICLC, CpG, and other TLR ligands.

### BRIEF SUMMARY OF THE DRAWINGS

FIGS. 1A-1E: Nucleic acid (DNA) polycistronic vaccine constructs designed for providing enhanced adaptive immune responses. (A) Schematic diagram of a DNA vaccine construct; (B) DNA vaccines are in nine formulations with selective antigen sequence repeated three times as independent cistrons that differ by the addition of specific sequences—a destabilization domain (D.D.), a lysosome-associated membrane protein domain (LAMP), and a signal sequence (s.s.)—that dictate the processing and presentation of the antigen protein. (C) DNA vaccines are in six formulations that comprise destabilization domain (D.D.) and LAMP domain, with omission of the signal sequence domain. (D) DNA vaccines are in six formulations that comprise the destabilization domain (D.D.) and signal sequence (s.s.), with omission of the LAMP domain. (E) DNA vaccine is in two formulations that comprise the LAMP and signal sequence domains, omitting the destabilization domain (D.D.).

FIG. 2A-2E: Schematic diagram of an mRNA vaccine construct. The coding region is flanked by sequences corresponding to a 5′ 7-methylguanosine triphosphate (m⁷G) cap and 5′ untranslated region (5′ UTR) at the 5′ end, and 3′ untranslated region (3′UTR) that includes a poly(A) tail, and optionally a 3′-immunoenhancing element (IE) at the 3′ end. FIGS. 2B-2E depict ten exemplary polycistronic RNA vaccine constructs with selective (target) antigen sequence repeated two or three times as independent cistrons that differ by the addition of specific sequences—a destabilization domain (D.D.), a lysosome-associated membrane protein domain (LAMP), and a signal sequence (s.s.)—that dictate the processing and presentation of the antigen protein designed for providing enhanced adaptive immune responses.

FIG. 3: Amino acid sequence of an exemplary destabilization domain (D.D.) for MHC-1 (CTL) activation.

FIG. 4: Amino acid (aa) sequence of an exemplary LAMP domain (417 aa) for MHC-II (HTL) activation. (aa residues 1-382: lumenal domain; aa residues 383-417: transmembrane domain and cytoplasmic tail.)

FIG. 5: Amino acid sequence of an exemplary signal sequence (24 aa).

FIG. 6: The nucleotide sequence of internal ribosome entry site (IRES) from encephalomyocarditis virus (575 bases).

FIG. 7: Interleukin-2 (IL-2) response of mice immunized with exemplary polycistronic constructs encoding the ovalbumin (OVA) antigen as measured by ELISA assay. The y-axis shows IL-2 level after stimulation of OVA (pg/ml) and the x-axis shows the constructs: (1) s.s. OVA; (2) LAMP/OVA; (3) OVA/D.D.; (4) DNA mixture of LAMP/OVA and OVA/D.D.; (5) DNA mixture of LAMP/OVA and OVA/D.D. and s.s. OVA; (6) polycistronic LAMP/OVA-IRES-OVA/D.D.

FIG. 8: Interferon (IFN) gamma response of mice immunized with exemplary polycistronic constructs encoding the ovalbumin (OVA) antigen measured by ELISA assay. The y-axis shows IFN gamma level after stimulation with OVA (pg/ml) and the x-axis shows the constructs: (1) LAMP/OVA; (2) OVA/D.D.; (3) DNA mixture of LAMP/OVA and OVA/D.D.; (4) DNA mixture of LAMP/OVA and OVA/D.D. and s.s. OVA; (5) Polycistronic LAMP/OVA-IRES-OVA/D.D.; (6) polycistronic LAMP/OVA-IRES-OVA/D.D.-IRES-s.s.OVA.

FIG. 9: Granzyme B response as measured by ELISA assay of mice immunized with exemplary polycistronic constructs encoding the ovalbumin (OVA) antigen. The y-axis shows Granzyme B level after stimulation of OVA (pg/ml) and the x-axis shows the constructs: (1) LAMP/OVA; (2) OVA/D.D.; (3) DNA mixture of LAMP/OVA and OVA/D.D.; (4) DNA mixture of LAMP/OVA and OVA/D.D. and s.s. OVA; (5) polycistronic LAMP/OVA-IRES-OVA/D.D.; (6) polycistronic LAMP/OVA-IRES-OVA/D.D.-IRES-s.s.OVA.

FIG. 10: Interleukin-10 (IL-10) response of mice immunized with exemplary polycistronic constructs encoding the ovalbumin (OVA) antigen as measured by ELISA assay. The y-axis shows Interleukin-10 (IL-10) level after stimulation of OVA (pg/ml) and the x-axis shows the constructs: (1) LAMP/OVA; (2) OVA/D.D.; (3) DNA mixture of LAMP/OVA and OVA/D.D.; (4) DNA mixture of LAMP/OVA and OVA/D.D. and s.s. OVA; (5) polycistronic LAMP/OVA-IRES-OVA/D.D.; (6) polycistronic LAMP/OVA-IRES-OVA/D.D.-IRES-s.s.OVA.

FIG. 11: Interleukin-6 (IL-6) response of mice immunized with exemplary polycistronic constructs encoding the ovalbumin (OVA) antigen as measured by ELISA assay. The y-axis shows Interleukin-6 (IL-6) level after stimulation of OVA (pg/ml) and the x-axis shows the constructs: (1) LAMP/OVA; (2) OVA/D.D.; (3) DNA mixture of LAMP/OVA and OVA/D.D.; (4) DNA mixture of LAMP/OVA and OVA/D.D. and s.s. OVA; (5) polycistronic LAMP/OVA-IRES-OVA/D.D.; (6) polycistronic LAMP/OVA-IRES-OVA/D.D.-IRES-s.s.OVA.

FIG. 12: Interleukin-4 (IL-4) response of mice immunized with exemplary polycistronic constructs encoding the ovalbumin (OVA) antigen as measured by ELISA assay. The y-axis shows Interleukin-4 (IL-4) level after stimulation of OVA (pg/ml) and the x-axis shows the constructs: (1) LAMP/OVA; (2) OVA/D.D.; (3) DNA mixture of LAMP/OVA and OVA/D.D.; (4) DNA mixture of LAMP/OVA and OVA/D.D. and s.s. OVA; (5) polycistronic LAMP/OVA-IRES-OVA/D.D.; (6) polycistronic LAMP/OVA-IRES-OVA/D.D.-IRES-s.s.OVA.

FIG. 13: Interleukin-5 (IL-5) response of response of mice immunized with exemplary polycistronic constructs encoding the ovalbumin (OVA) antigen as measured by ELISA assay. The y-axis shows Interleukin-5 (IL-5) level after stimulation of OVA (pg/ml) and the x-axis shows the constructs: (1) LAMP/OVA; (2) OVA/D.D.; (3) DNA mixture of LAMP/OVA and OVA/D.D.; (4) DNA mixture of LAMP/OVA and OVA/D.D. and s.s. OVA; (5) polycistronic LAMP/OVA-IRES-OVA/D.D.; (6) polycistronic LAMP/OVA-IRES-OVA/D.D.-IRES-s.s.OVA.

FIGS. 14A-D: Exemplary nucleic acid constructs designed to enhance human dendritic cell vaccine-induced T lymphocyte responses. (A) CMV pp65 antigen (Cytomegalovirus, CMV). (B) D.D.-CMV pp65. (C) CMV pp65-LAMP. (D) D.D.-CMV pp65-LAMP.

FIG. 15: Flow cytometric measurement of phenotype of dendritic cells. PBMCs from healthy donors were cultured in 37° C., 5% CO2 incubator for 2 hours. The adherent cells were then stimulated by 8001 U/ml GM-CSF and 5001 U/ml IL-4 in AIM-V media for 6 days to generate immature dendritic cells (iDC). On day 6, 160 ng/ml IL6, 5 ng/ml TNF-α, 5 ng/ml IL-10 and 1 ug/ml PGE2 were added. On day 7, mature dendritic cells (mDC) were harvested. The phenotype of immature dendritic cells (iDC) and mature dendritic cells (mDC) (CD14, CD11c, CD80, CD83, CD86, CCR7, HLA-ABC and HLA-DR) were measured by flow cytometry.

FIGS. 16A-B: Expression levels of CMV pp65 antigen in dendritic cells. D.D.-CMV pp65 mRNA or CMV pp65 mRNA was transfected into dendritic cells respectively by electroporation. Then, the expression levels of CMV pp65 antigen in dendritic cells were measured by flow cytometry. (A) A representative FACS Graph. (B) A summary wave, n=3. No dramatic difference was observed between D.D.-CMV pp65 group and CMV pp65 group. (p>0.05).

FIGS. 17A-D: CMV pp65 specific T cell responses. PMBCs from healthy donors were stimulated twice on day 0 and day 7 by D.D.-CMV pp65 mRNA or CMV pp65 mRNA loaded dendritic cells respectively. (A) CD8 T cell IFN-γ, TNF-α and CD4 T cell IFN-γ responses were measured on day 14 by flow cytometry. (B-D) Summary of CD8 T cell IFN-γ, TNF-α or CD4 T cell IFN-γ response. Paired-Samples T Test was used. n=6.

FIG. 18: CMV pp65 specific T cell responses induced by mRNA antigen-loaded mature dendritic cells (mDC). PMBCs from healthy donors were stimulated twice on day 0 and day 7 by D.D.-CMV pp65 mRNA, CMV pp65-LAMP mRNA, Mix (D.D.-CMV pp65: CMV pp65-LAMP=1:1) mRNA or D.D.-CMV pp65-LAMP mRNA loaded mDC respectively. CD8 T cells IFN-γ, TNF-α and CD4 T cells IFN-γ responses were measured on day 14 by flow cytometry.

FIG. 19: CMV pp65 specific T cell responses induced by mRNA antigen-loaded iDC. D.D.-CMVpp65 mRNA, CMVpp65-LAMP1 mRNA, Mix (D.D.-CMV pp65: CMV pp65-LAMP1=1:1) mRNA or D.D.-CMVpp65-LAMP1 mRNA was transfected into immature dendritic cells (iDC) respectively. Then immature dendritic cells (iDC) were further cultured to be mature dendritic cells (mDC). PMBCs from healthy donors were stimulated twice on day 0 and day 7 by mRNA loaded mDC above. CD8 T cells IFN-γ, TNF-α and CD4 T cells IFN-γ responses were measured on day 14 by flow cytometry.

FIG. 20A-C: Human gp100 specific T cell responses induced by mRNA antigen-loaded DC. Human gp100, LAMP-gp100, DD-gp100, SS-gp100 or LAMP-gp100-IRES-DD-gp100-IRES-SS-gp100 mRNA was transfected into immature dendritic cells (iDC) respectively. Then immature dendritic cells (iDC) were further cultured to be mature dendritic cells (mDC). PBMCs from healthy donors were stimulated three times on day 0, day 7 and day 13 by mRNA loaded mDC above. During the cell culture process, 1 ug/ml anti-human-PD-L and PD-L2 antibodies were added. CD3+ T cell TNF-α& IFN-γ, CD8 T cell TNF-α& IFN-γ and CD4 T cell IFN-γ responses were measured on day 14 by flow cytometry.

FIG. 21: Expression of nanoparticles delivered CMV-pp65 mRNA in DC. CMV-pp65 mRNA or mock control was transfected into DC by nanoparticles delivery system, in a concentration of 1 μg mRNA/1×10⁵ cells. DCs were cultured in 37° C. 5% CO₂ incubator and harvested at 6 h, 12 h and 24 h. Duplicated wells were set for each condition. The expression of CMV-pp65 in DC was measured by flow cytometry.

FIG. 22A-B: Enhanced MHC class I epitope presentation by coupling OVA to Destabilizing Domain (DD). Flow cytometry analysis of BMDCs stained with 25D1.16 Ab to measure SIINFEKL/H2-Kb complexes after transfection with p43-ova, p43-SS/ova, p43-DD/ova or p43-mLamp/ova 24 h. (A) Representative contour plots and median fluorescence intensities (MFI) were shown and individual percentage is depicted. (B) MFI data represent mean values of three independent experiments±SEM.

FIG. 23A-B: Comparison of the effect of the DD-modification method with other methods on MHC-1/peptide antigen presentation. Flow cytometry analysis of BMDCs stained with 25D1.16 Ab to measure SIINFEKL/H2-Kb complexes after transfection with p43-DD/ova, p43-GTN/ova, p43-P62/ova or p43-UBT/ova 24 h after transfection. (A) Representative contour plots and median fluorescence intensities (MFI) were shown and individual percentage is depicted. (B) MFI data represent mean values of three independent experiments±SEM.

FIG. 24: Anti-tumor immunity mediated by different forms of OVA antigen. B16/F10/mOVA melanoma cells (5×10⁴/mouse) were s.c. inoculated into right flank of C57BL/6 mice on day 0. In single therapy group mice were then immunized by i.p. injection of PBS or 1×10⁶ DCs electroporated with p43-ova, p43-DD/ova or p43-mLAMP/ova on day 7 and 14. In combination therapy group, 5×10⁵ DCs electroporated with p43-DD/ova and 5×10⁵ DCs electroporated with p43-mLAMP/ova were injected. Tumor growth was monitored daily starting from day 5. Shown are tumor diameter and weight in these mice (n=1-5 mice per group).

### DETAILED DESCRIPTION OF THE INVENTION

Unless defined otherwise, all technical and scientific terms used herein have the same meaning as commonly understood by one of ordinary skill in the art to which the disclosure is related. For example, The Concise Dictionary of Biomedicine and Molecular Biology, Juo, Pei-Show, 2nd ed. 2002, CRC Press; The Dictionary of Cell and Molecular Biology, 3rd ed., 1999, Academic Press; and The Oxford Dictionary of Biochemistry and Molecular Biology, Revised 2000, Oxford University Press, provide one of skill in the art with a general dictionary of many of the terms used herein. Additionally, commonly used molecular biology terms, methods and protocols are provided in Molecular Cloning: A laboratory manual, M. R. Green and J. Sambrook (eds.), 4^{th} ed. 2012, Cold Spring Harbor Laboratory Press, New York. Additional definitions are set forth throughout the detailed description.

The present invention generally relates to vaccine design, polycistronic vaccine constructs (DNA, RNA, peptides, protein), vaccine compositions, and methods of use thereof as designed for enhanced antigen-specific vaccination. In particular, the polycistronic vaccine constructs provided by the invention express at least one target antigen, and comprise a plurality of independent cistrons, wherein each independent cistron encodes a modified target antigen comprising an in-frame fusion protein of the target antigen and at least one specific domain that dictates the processing and presentation of the antigen. In certain embodiments, the specific domain comprises a destabilization domain (D.D.), a lysosome-associated membrane protein (LAMP) domain and a signal sequence (s.s.). The polycistronic vaccine constructs provided by the present invention can comprise any target antigen(s), and accordingly, the vaccines provided by the present invention (DNA, RNA, or protein) can be used to modulate or enhance an immune response against any kind of antigen. The present invention also provides methods of use thereof for eliciting an enhanced activation of each of the three arms of the adaptive immune response: CD8+ cytolytic T lymphocyte (CTL), CD4+ helper T lymphocyte (HTL) and antibody, by virtue of the specific functionalities conferred by the specific domains, e.g., LAMP, D.D., and s.s., domains. Further, the present invention provides methods of use of the mRNA encoded by the polycistronic constructs to enhance dendritic cell (D.C.) vaccine-induced T-cell responses. The present invention also provides methods for cellular therapy comprising an engineered dendritic cell (e.g., mRNA loaded dendritic cell).

In some aspects, the present invention provides nucleic acid vaccines (DNA and RNA/mRNA) comprising or encoded by the polycistronic vaccine constructs of the invention. Nucleic acid vaccines are vaccines containing antigens encoded by either DNA or RNA (mRNA). In certain embodiments, the nucleic acid vaccines are provided as a vaccine composition. The polycistronic DNA vaccine constructs provided by the invention are administered to a host (subject) and internalized by host cells, where it is transcribed in the nucleus and translated in the cytoplasm by host cellular functions. The resulting proteins are processed in the context of D.D., LAMP, or secreted constructs by which CTL and HTL antigen sequences are ultimately presented on the surface of host antigen-presenting cells (APC) in the context of major histocompatibility complex (MHC) molecules. This can occur by APC being directly transfected with the DNA or by cross-presentation from non-APC to APC. The peptide-MHC complex is recognized by antigen-specific T cells, resulting in a cellular host immune response. Protein products targeted for secretion are directed to the surface of the transfected cell where they are secreted for activation of B-cells and antibody synthesis. The polycistronic RNA vaccines provided by the invention comprise messenger RNA (mRNA) synthesized by in vitro transcription (IVT) using a bacteriophage RNA polymerase from the polycistronic constructs or mRNAs that are synthesized artificially. Once administered and internalized by host cells, the mRNA transcripts are translated directly in the cytoplasm and then, like DNA vaccines, the resulting antigens are presented to APC to stimulate an immune response.

With respect to DNA and RNA vaccines, a major advance has been the use of a lysosome-associated membrane protein (LAMP) domain. The LAMP protein is co-localized with the MHCII protein in the endosomal/lysosomal compartment of professional antigen presenting cells and vaccines with pathogen sequences synthesized as a chimera of the lumenal domain of LAMP have greatly enhanced trafficking to this compartment where the antigenic domain is processed and peptides from it are presented on the cell surface in association with major histocompatibility (MHC) class II molecules (MHC-II), thereby enhancing CD4⁺ T cell activation (see, for example, U.S. Pat. Nos. 5,633,234; 8,318,173; 8,445,660; and 9,499,589, each of which is incorporated herein in its entirety).

However, a major limitation of existing vaccination technology is the lack of stimulation of class I MHC-mediated (MHC-I) and activation of CD8⁺ cytolytic T cell response (CTL). Aspects of the present invention address this challenge by providing polycistronic vaccine constructs comprising, inter alia, a destabilization domain (D.D.) to facilitate processing, by the proteasome, of a modified (fused) antigen, thereby enhancing MHC-I presentation of the antigen. This leads to stimulation of CD8⁺ CTL response. In particular, the polycistronic design of the vaccine constructs provided by the present invention, and its encoded mRNA, confers the advantage of simultaneously activating each of the three arms of adaptive immune response using a single construct.

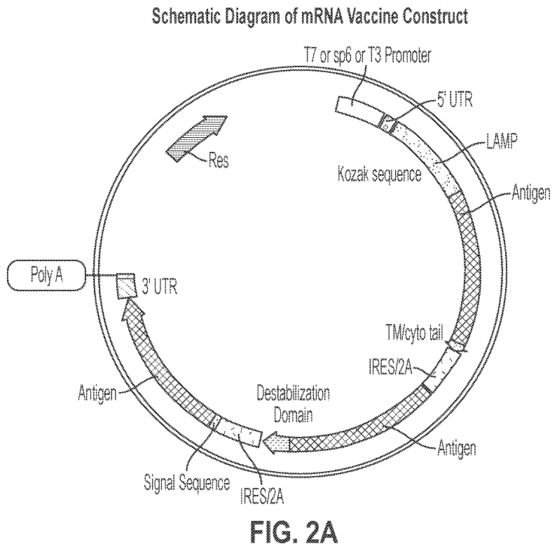

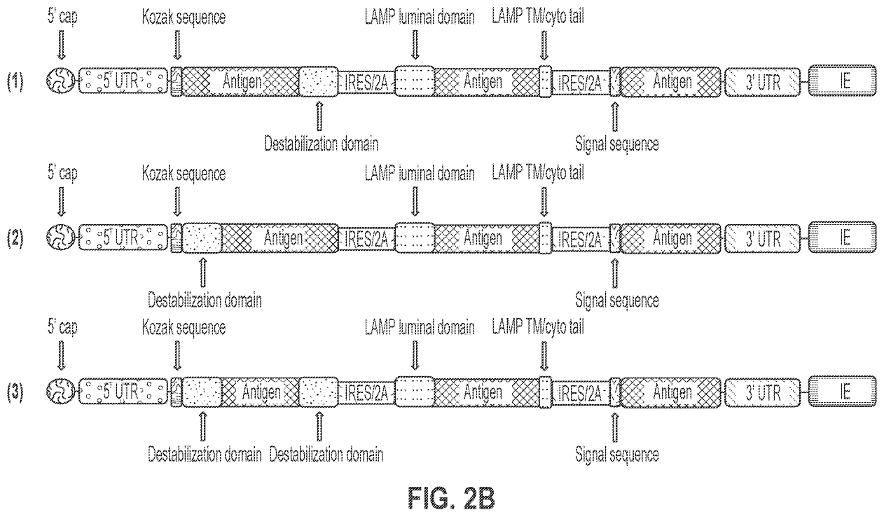

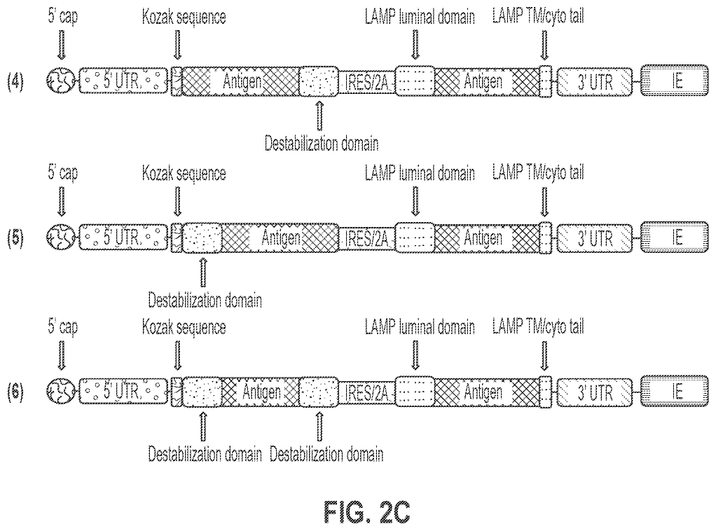

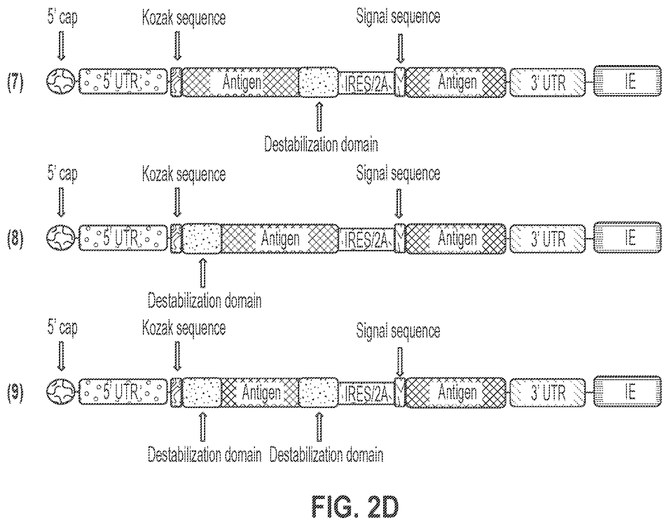

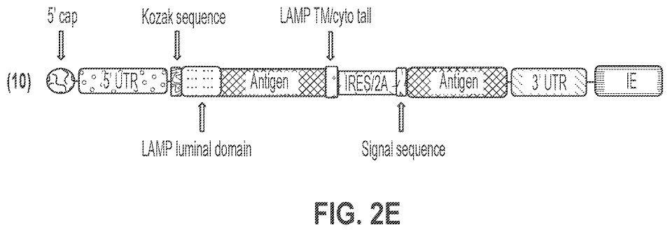

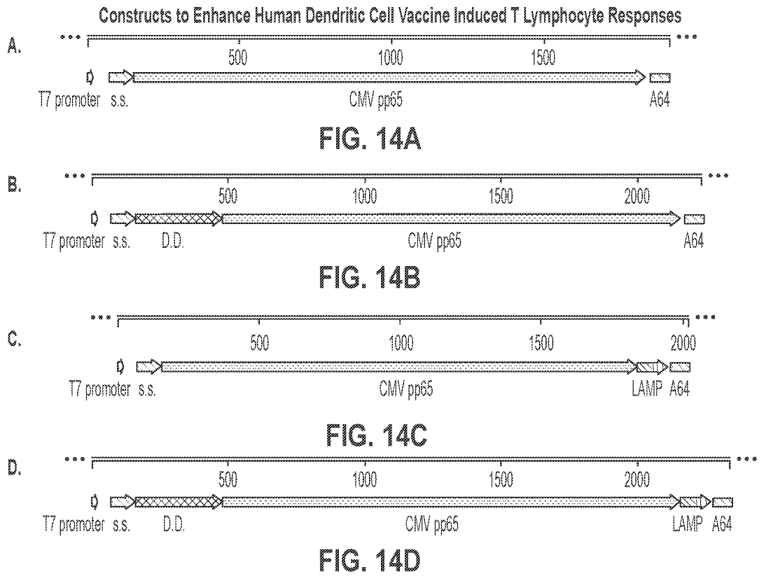

In some aspects, the present invention provides mRNA-based antigen presenting cells (APCs), for example, mRNA-based dendritic cells (engineered dendritic cells) and dendritic cell vaccine compositions comprising one or more of the polycistronic vaccine constructs (e.g., FIGS. 2A-2E) or the fusion constructs (e.g., FIGS. 14A-D) provided by the present invention.

In some aspects, the present invention provides a method for modulating an immune response in a subject comprising administering any of the polycistronic vaccine constructs or vaccine compositions provided by the invention. In some aspects, the present invention provides a method for providing enhanced antigen-specific vaccination in a subject comprising administering any of the polycistronic vaccine constructs or vaccine compositions provided by the invention. In some aspects, the present invention provides a method for inducing a therapeutic immune response against a target antigen derived from a pathogen, a human self-protein or a malignant neoplasm comprising administering any of the polycistronic vaccine constructs or vaccine compositions provided by the invention.

In some aspects, the present invention provides a method of manufacturing mRNA-loaded antigen presenting cells (APCs), for example, a method of manufacturing mRNA-loaded dendritic cells, the method comprising the steps of: (a) providing dendritic cells; and (b) transfecting the immature dendritic cells with one or more messenger RNA (mRNA) species transcribed in vitro from the polycistronic nucleic acid vector constructs provided by the invention. In some aspects, the present invention provides a method enhancing vaccine-induced T-lymphocyte responses comprising administering to a subject in need thereof, a composition comprising an isolated dendritic cell comprising one or more messenger RNA (mRNA) species transcribed in vitro from the polycistronic nucleic acid constructs provided by the invention. In other aspects, the present invention provides a method enhancing vaccine-induced T-lymphocyte responses comprising administering to a subject in need thereof, a composition comprising a first isolated dendritic cell and a second isolated dendritic cell each comprising one or more messenger RNA (mRNA) species transcribed in vitro from the polycistronic vaccine constructs or nucleic acid constructs provided by the invention. In particular aspects, the first and the second isolated dendritic cells comprise different messenger RNA (mRNA) species or nucleic acid constructs provided by the invention. In certain embodiments, viral vectors (e.g., adenovirus, lentivirus, gamma-retrovirus) or bacterial vectors (e.g., Listeria monocytogenes, Salmonella typhimurium) incorporated with the DNA-encoding antigen expression cassette/construct can also be used to delivery antigen to dendritic cells or directly to patients.)

In some aspects, the present invention provides a packaged article, e.g., an article of manufacture, such as a kit or a system comprising any of the vaccine constructs, vaccine compositions, cells or any component(s) relating to any of the methods provided by the invention (e.g., methods for administration and delivery of the vaccine compositions described herein). The packaged article can include, optionally, a label(s) and/or instructions for use. Such instructions include directing or promoting, including advertising, use of said article of manufacture.

### Nucleic Acids, Vectors, and Proteins

As used herein, the terms “nucleic acid”, “polynucleotide”, “polynucleotide molecule”, “polynucleotide sequence” and plural variants are used interchangeably to refer to a wide variety of molecules, including single strand and double strand DNA and RNA molecules, cDNA sequences, genomic DNA sequences of exons and introns, chemically synthesized DNA and RNA sequences, and sense strands and corresponding antisense strands. Polynucleotides of the invention may also comprise known analogs of natural nucleotides that have similar properties as the reference natural nucleic acid.

Polynucleotides of the present invention may be cloned, synthesized, altered, mutagenized, or combinations thereof. Standard recombinant DNA and molecular cloning techniques used to isolate and modify nucleic acids are known in the art. Site-specific mutagenesis to create base pair changes, deletions, or small insertions is also known in the art (see e.g., M. R. Green and J. Sambrook (eds.) Molecular Cloning: A Laboratory Manual, 4^{th} ed., 2012, Cold Spring Harbor Laboratory Press, Cold Spring Harbor, New York; Silhavy et al., Experiments with Gene Fusions, 1984, Cold Spring Harbor Laboratory Press, Cold Spring Harbor, New York; Glover & Hames, DNA Cloning: A Practical Approach, 2nd ed., 1995, IRL Press at Oxford University Press, Oxford/New York; Ausubel (ed.) Short Protocols in Molecular Biology, 3rd ed., 1995, Wiley, New York).

As used herein, a polynucleotide or polynucleotide region (or a polypeptide or polypeptide region) which has a certain percentage (for example, at least about 50%, at least about 60%, at least about 70%, at least about 80%, at least about 85%, at least about 90%, at least about 95%, at least about 99%) of “sequence identity” to another sequence means that, when maximally aligned, using software programs routine in the art, that percentage of bases (or amino acids) are the same in comparing the two sequences.

Two sequences are “substantially homologous” or “substantially similar” when at least about 50%, at least about 60%, at least about 70%, at least about 75%, and at least about 80%, and at least about 90% or at least about 95% of the nucleotides match over the defined length of the DNA sequences. Similarly, two polypeptide sequences are “substantially homologous” or “substantially similar” when at least about 50%, at least about 60%, at least about 66%, at least about 70%, at least about 75%, and at least about 80%, and at least about 90% or at least about 95% of the amino acid residues of the polypeptide match over a defined length of the polypeptide sequence. Sequences that are substantially homologous can be identified by comparing the sequences using standard software available in sequence data banks. Substantially homologous nucleic acid sequences also can be identified in a Southern hybridization experiment under, for example, stringent conditions as defined for that particular system. Defining appropriate hybridization conditions is within the skill of the art.

In the context of nucleic acid sequences, the term “conservatively modified variant” refers to those nucleic acids which encode identical or essentially identical amino acid sequences, or where the nucleic acid does not encode an amino acid sequence, to essentially identical sequences. Specifically, degenerate codon substitutions can be achieved by generating sequences in which the third position of one or more selected (or all) codons is substituted with mixed-base and/or deoxyinosine residues (Batzer, et al. (1991) Nucleic Acid Res. 19: 5081; Ohtsuka, et al. (1985) J. Biol. Chem. 260: 2605-2608; Rossolini et al. (1994) Mol. Cell. Probes 8: 91-98).

The term “vector” or “expression vector” is used herein for the purposes of the specification and claims, to mean vectors used in accordance with the present invention as a vehicle for introducing into and expressing a desired gene product (e.g., antigen) in a cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

“Expression vector” means an engineered nucleic acid (DNA) construct containing at least one promoter operably linked to a downstream gene, cistron or RNA coding region. In the polycistronic vaccine constructs of the present invention, the promoter can be operably linked to one or more genes or cistrons each initiated by a start and followed by a stop codon. Transfection of the expression vector into a recipient cells, i.e., eukaryotic cell, e.g., mammalian cell, fungal cell, yeast cell, allows the cell to express antigens encoded by the expression vector. Expression vectors include e.g., plasmid vectors, and viral vectors. Expression vector constructs provided by the present invention include chimeric (fusion) constructs and polycistronic vector constructs.

As used herein, a “viral vector” refers to a virus or viral particle that comprises a polynucleotide to be delivered into a host cell, either in vivo, ex vivo or in vitro. Examples of viral vectors include, but are not limited to, adenovirus vectors, adeno-associated virus vectors, and retroviral vectors. In certain aspects where gene transfer is mediated by an adenoviral vector, a vector construct refers to the polynucleotide comprising the adenovirus genome or part thereof, and a selected, non-adenoviral gene, in association with adenoviral capsid proteins.

As used herein, “operably linked” or “under transcriptional control” refers to expression (e.g., transcription or translation) of a polynucleotide sequence which is controlled by an appropriate juxtaposition of an expression control element and a coding sequence. In some aspects, a DNA sequence is “operatively linked” to an expression control sequence, in the 5′ to 3′ orientation, when the expression control sequence controls and regulates the transcription of that DNA sequence.

“Promoter” means a minimal sequence sufficient to direct transcription, in a prokaryotic or a eukaryotic cell. The definition includes promoter elements that are sufficient to render promoter-dependent gene expression controllable in a cell type-specific, tissue-specific, or temporal-specific manner, or inducible by external signals or agents, such elements may be located in the 5′ or 3′ or intron sequence regions of a particular gene. Exemplary promoters for use in the invention include, but are not limited to viral, mammalian, bacteriophage, and yeast promoters that provide for high levels of expression, e.g., mammalian cytomegalovirus (CMV) promoter, Rous sarcoma virus (RSV) promoter, elongation factor-1 alpha (EF1α) promoter, CMV early enhancer/chicken R actin (CAG) promoter, Ubiquitin C (UbC) promoter, MCi promoter, R Actin promoter, yeast alcohol oxidase, phosphoglycerokinase (PGK) promoter, lactose inducible promoters, galactosidase promoter, adeno-associated viral promoter, baculovirus promoter, poxvirus promoter, retroviral promoters, adenovirus promoters, SV40 promoter, HMG (hydroxymethylglutarylcoenzyne A) promoter, TK (thymidine kinase) promoter, 7.5K or H5R poxvirus promoters, adenovirus type 2 MPC late promoter, alpha-antrypsin promoter, factor IX promoter, immunoglobulin promoter, CFTR surfactant promoter, albumin promoter, transferrin promoter, bacteriophage T3 promoter, bacteriophage T7 promoter, and SP6 promoter. In addition to the promoter, plasmids used in the present invention can comprise additional regulatory elements such as adenovirus IRT elements to enhance immune responses, as well as a strong polyadenylation/transcriptional termination signal, such as bovine growth hormone or rabbit beta-globulin polyadenylation sequences.

“Cistron” means a “coding sequence” or sequence of nucleic acid that encodes a single protein or polypeptide.

As used herein, the terms “polycistronic vector,” “polycistronic expression vector,” “polycistronic vector construct,” or “polycistronic vaccine construct” refer to an expression vector which allows the simultaneous expression of two and more distinct gene products (e.g., antigens) encoded by two or more distinct (independent) cistrons from a single transcript (i.e., a polycistronic mRNA).

As used herein, the terms “polypeptide,” “protein” and plural variants are used interchangeably and refer to a compound made up of a single chain of amino acids joined by peptide bonds. Polypeptides of the invention may comprise naturally occurring amino acids, synthetic amino acids, genetically encoded amino acids, non-genetically encoded amino acids, and combinations thereof. Polypeptides may include both L-form and D-form amino acids. Polypeptides may include a “biologically active fragment”, a “biologically active form”, a “biologically active equivalent” of and a “functional derivative” of a wild-type protein, possesses a biological activity that is at least substantially equal (e.g., not significantly different from) the biological activity of the wild type protein as measured using an assay suitable for detecting the activity.

Isolated polypeptides of the invention may be purified and characterized using a variety of standard techniques that are known to the skilled artisan (see e.g., Schröder et al., The Peptides, 1965, Academic Press, New York; Bodanszky, Principles of Peptide Synthesis, 2nd rev. ed. 1993, Springer-Verlag, Berlin/New York; Ausubel (ed.), Short Protocols in Molecular Biology, 3rd ed., 1995, Wiley, New York).

### Polycistronic Vaccine Constructs

In some aspects, the vaccine constructs provided by the present invention express at least one target antigen to which an immune response is desired, wherein the construct comprises a plurality of independent cistrons operably linked to a single promoter, in the 5′ to 3′ orientation, wherein each independent cistron encodes a modified target antigen comprising an in-frame fusion protein of the target antigen and at least one specific domain that dictates the processing and presentation of the antigen.

As used herein, “target antigen,” “immunogen,” or “antigenic material,” means a molecule or substance, including a fragment, epitope or derivative thereof, and further includes fused polypeptides from one or more source proteins (e.g., in-frame fusions of multiple antigens separated by a polypeptide linker rich in glycine), that induces a specific immune response in a host. An “epitope,” “antigenic epitope,” “antigenic fragment,” or “immunoreactive fragment” may be used interchangeably, and is defined a structure, usually made up of a short peptide sequence or oligosaccharide that is specifically recognized or specifically bound by a component of the immune system. As used herein, a “modified target antigen” refers to a modification made to a target antigen by fusing (in-frame) one or more antigenic (immunogenic) sequences to one or more other sequences such as a functional domain (e.g., LAMP, D.D., s.s.) so as to modify its immunogenicity. In some embodiments, the polycistronic vaccine construct can comprise two target antigens, three target antigens, four target antigens, five target antigens, six target antigens, seven target antigens, eight target antigens, nine target antigens, ten target antigens or more than ten target antigens. The polycistronic vaccine constructs described herein can encompass any target antigen, including but not limited to an antigen derived from a pathogen, a human self-protein, or a tumor antigen (including a malignant neoplasm). The term “tumor antigen” includes any antigenic substance produced in a tumor cell, which triggers an immune response in a host. The term tumor antigen encompasses, e.g., tumor-specific antigens (TSA), tumor-associated antigens (TAA), neoantigens, tissue differentiation antigens, mutant protein antigens, oncogenic viral antigens, cancer-testis antigens and vascular or stromal specific antigens. Exemplary target antigens, include without limitation, any tumor antigen, e.g., neoantigens identified from a patient using genomic sequencing, human gp100 tumor antigen, transplantation antigens, cell surface proteins found on mammalian cells, cancer-specific proteins, proteins associated with abnormal physiological responses, proteins of bacteria, protozoa or fungi, including especially proteins found in the cell walls or cell membranes of these organisms, and proteins encoded by the genomes of viruses including retroviruses such as HIV and hepadnaviruses, viral antigens (derived from infectious viruses), influenza virus hemagglutinin (HA protein), synthetic antigens (e.g., synthetic antigenic peptide epitopes) altered antigens, and mixtures, combinations, derivatives, antigenic fragments of any the above target antigens, which are immunogenic.

Additional exemplary target antigens within the scope of the present invention include without limitation, antigens encoded by the genomes of organisms causative for, or associated with, hepatitis, rabies, malaria (e.g., epitopes displayed by Plasmodium falciparum), parasitic infections (e.g., such as schistosomiasis), cancer, AIDS, yellow fever, dengue fever, Japanese encephalitis, West Nile fever, measles, smallpox, anthrax, Ebola, equine encephalitis, Rift valley fever, cat scratch fever, viral meningitis, plague, tularemia, and diseases caused by other pathogenic organisms. Viral antigens include virally-encoded proteins encoded by the genome of viruses pathogenic to man, horses, cows, pigs, llamas, giraffes, dogs, cats or chickens. Non-limiting examples include peptides from the influenza nucleoprotein composed of residues 365-80 (NP365-80), NP50-63, and NP147-58 and peptides from influenza hemagglutinin HA202-21 and HA523-45. Other exemplary antigens include, but are not limited to, an HIV encoded polypeptide such as Gag, Env, Rev, Tat, and/or Nef polypeptides, gp160, and the like; papilloma virus core antigen; HCV structural and non-structural proteins; and CMV structural and non-structural proteins.

Exemplary tumor antigens within the scope of the present invention include, but are not limited to, 5T4, AIM2, AKAP4 2, Art-4, Aura A1 (AURKA), Aura B1 (AURKB), BAGE, BCAN, B-cyclin, BSG, CCND1, CD133, CDC45L, CDCA1 (TTK), CEA, CHI3L2 (Chitinase 3-like 2), CSPG4, EpCAM 4, Epha2, EPHX1, Ezh2, FABP7, Fos11 (Fra-1), GAGE, Galt-3, G250 (CA9), gBK, glast, GnT-V, gp100, HB-EGF, HER2, HNPRL, HO-1, hTERT, IGF2BP3, IL13-Ra2, IMP-3, IQGAP1, ITGAV, KIF1C, KIF20A, KIF21B, KIFC3, KK-LC-1, LAGE-1, Lck, LRRC8A, MAGE-1 (MAGEA1), MAGE-2 (MAGEA2B), MAGE-3, MAGE-4, MAGE-6, MAGE-10, MAGE-12, MAGE-C1 (CT7), MAGE-C2, MAGE-C3, Mart-1, MELK, MRP3, MUC1, NAPSA, NLGN4X, Nrcam, NY-ESO-1 (CTAGIB), NY-SAR-35, OFA/iLRP, PCNA, PIK3R1, Prame, PRKDC, PTH-rP, PTPRZ1, PTTG1 2, PRKDC, RAN, RGS1, RGS5, RHAMM (RHAMM-3R), RPL19, Sart-1, Sart-2, Sart-3, SEC61G, SGT-1, SOX2, Sox10, Sox11, SP17, SPANX-B, SQSTM1, SSX-2, STAT1, STAT3, Survivin, TARA, TNC, Trag-3, TRP-1, TRP2, Tyrosinase, URLC10 (LY6K), Ube2V, WT1, XAGE-lb (GAGED2a), YKL-40 (CHI3L1), ACRBP, SCP-1, SSX-1, SSX-4, NY-TLU-57, CAIX, Brachyury, NY-BR-1, ErbB, Mesothelin, EGFRvII, IL-13Ra2, MSLN, GPC3, FR, PSMA, GD2, L1-CAM, VEGFR1, VEGFR2, KOC1, OFA, SL-701, Mutant P53, DEPDC1, MPHOSPH1, ONT-10, GD2L, GD3L, TF, PAP, BRCA1 DLC1, XPO1, HIF1A, ADAM2, CALR3, SAGE1, SCP-1, ppMAPkkk, WHSC, Mutant Ras, COX1, COX2, FOXP3, IDO1, ID02, TDO, PDL1, PDL2, and PGE2.

Exemplary neoantigens within the scope of the present invention include, but are not limited to, neoantigens associated with any tumor/cancer, e.g., lung cancer (MTFR2 D326Y, CHTF18 L769V, MYADM R30W, HERC1 P3278S, FAM3C K193E, CSMD1 G3446E, SLC26A7 R117Q, PGAP1 Y903F, HELB P987S, ANKRD K603T); melanoma (TMEM48 F169L, TKT R438W, SEC24A P469L, AKAP13 Q285K, EXOC8 Q656P, PABPCI R520Q, MRPS5 P59L, ABCC2 S1342F, SEC23A P52L, SYTL4 S363F, MAP3K9 E689K, AKAP6 M1482I, RPBM P42L, HCAPG2 P333L, H3F3C T4I, GABPA E161K, SEPT2 Q125R, SRPX P55L, WDR46 T300I, PRDX3 P101L, HELZ2 D614N, GCN1L1 P769L, AFMID A52V, PLSCR4 R247C, CENPL P79L, TPX2 H458Y, SEC22C H218Y, POLA2 L420F, SLC24A5 mut); Mesothelioma (NOTCH2 G703D, PDE4DIP L288M, BAP1 V523fs, ATP10B E210K, NSD1 K2482T); Glioma/Glioblastoma (IDH1 R132H, POLE L424V); Breast cancer (mPALB2, mROBO3, mZDHHCi6, mPTPRS, RBPJ H204L); Cholangiocarcinoma (ERBB2IP E805G); and Cervical cancer (MAPK1 E322K, PIK3CA E545K, PIK3CA E542K, EP300 D1399N, ERBB2 S310F, ERBB3 V104M, KRAS G12D). The neoantigens can comprise full length polypeptides (proteins) containing the neoantigenic epitope(s), or can be linked by generating fusion proteins or via a linker (e.g., 2A, IRES) as described herein for any target antigen, and incorporated into the polycistronic vaccine constructs provided by the present invention.

Further exemplary target antigens within the scope of the present invention include, but are not limited to, viral pathogens associated with the following infectious diseases: Acquired immunodeficiency syndrome (AIDS) (Human immunodeficiency virus (HIV)); Argentine Teagan fever (Junin virus); Astrovirus infection (Astroviridae family); BK virus infection (BK virus); Bolivian hemorrhagic fever (Machupo virus); Brazilian hemorrhagic fever (Sabii virus); Chickenpox (Varicella zoster virus (VZV)); Chikungunya (Alphavirus); Colorado tick fever (CTF) (Colorado tick fever virus (CTFV)); Common cold, Acute viral rhinopharyngitis, Acute coryza (usually rhinoviruses and coronaviruses); Cytomegalovirus infection (Cytomegalovirus); Dengue fever (Dengue viruses (DEN-1, DEN-2, DEN-3 and DEN-4) and other Flaviviruses including but not limited to West Nile virus (West Nile Fever), Yellow fever virus (Yellow fever); Zika virus (Zika fever) and tick-borne encephalitis virus; Ebola hemorrhagic fever (Ebolavirus (EBOV)); Enterovirus infection (Enterovirus species); Erythema infectiosum (Fifth disease) (Parvovirus B19); Exanthem subitum (Sixth disease) (Human herpesvirus 6 (HHV-6) and Human herpesvirus 7 (HHV-7)); Hand, foot and mouth disease (HFMD) (Enteroviruses, mainly Coxsackie A virus and Enterovirus 71 (EV71)); Hantavirus Pulmonary Syndrome (HPS) (Sin Nombre virus); Hepatitis A (Hepatitis A virus); Hepatitis B (Hepatitis B virus); Hepatitis C (Hepatitis C virus); Hepatitis D (Hepatitis D Virus); Hepatitis E (Hepatitis E virus); Herpes simplex (Herpes simplex virus 1 and 2 (HSV-1 and HSV-2)); Human bocavirus infection (Human bocavirus (HBoV)); Human metapneumovirus infection (Human metapneumovirus (hMPV)); Human papillomavirus (HPV) infection (Human papillomavirus (HPV)); Human parainfluenza virus infection (Human parainfluenza viruses (HPIV)); Epstein-Barr virus infectious mononucleosis (Mono) (Epstein-Barr virus (EBV)); human influenza viruses (influenza A, including but not limited to H1N1, H2N2, H3N2, H5N1, H7N9, influenza B, and other members of the Orthomyxoviridae family); Lassa fever (Lassa virus); Lymphocytic choriomeningitis (Lymphocytic choriomeningitis virus (LCMV)); Marburg hemorrhagic fever (MHF) (Marburg virus); Measles (Measles virus); Middle East respiratory syndrome (MERS) (Middle East respiratory syndrome coronavirus); Molluscum contagiosum (MC) (Molluscum contagiosum virus (MCV)); Monkeypox (Monkeypox virus); Mumps (Mumps virus); Norovirus (children and babies) (Norovirus); Poliomyelitis (Poliovirus); Progressive multifocal leukoencephalopathy (JC virus); Rabies (Rabies virus); Respiratory syncytial virus infection (Respiratory syncytial virus (RSV)); Rhinovirus infection (Rhinovirus); Rift Valley fever (RVF) (Rift Valley fever virus); Rotavirus infection (Rotavirus); Rubella (Rubella virus); Shingles (Herpes zoster) (Varicella zoster virus (VZV)); Smallpox (Variola) (Variola major or Variola minor); Subacute sclerosing panencephalitis (Measles virus); Venezuelan equine encephalitis (Venezuelan equine encephalitis virus); Venezuelan hemorrhagic fever (Guanarito virus); Viral pneumonia multiple viruses. Subjects suffering from or at risk of developing any of the above diseases will benefit from the compositions and methods described herein, and are within the scope of the present invention.

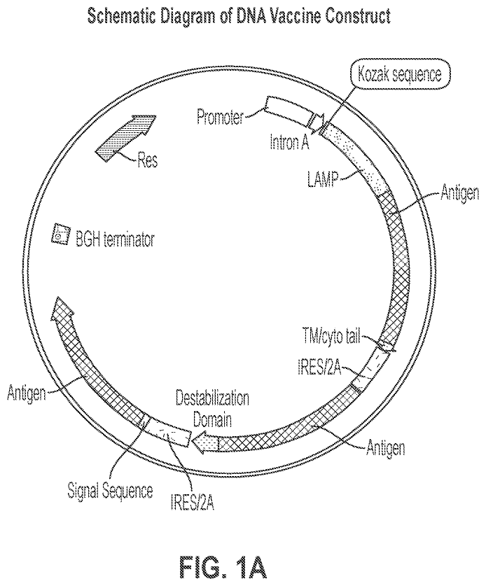

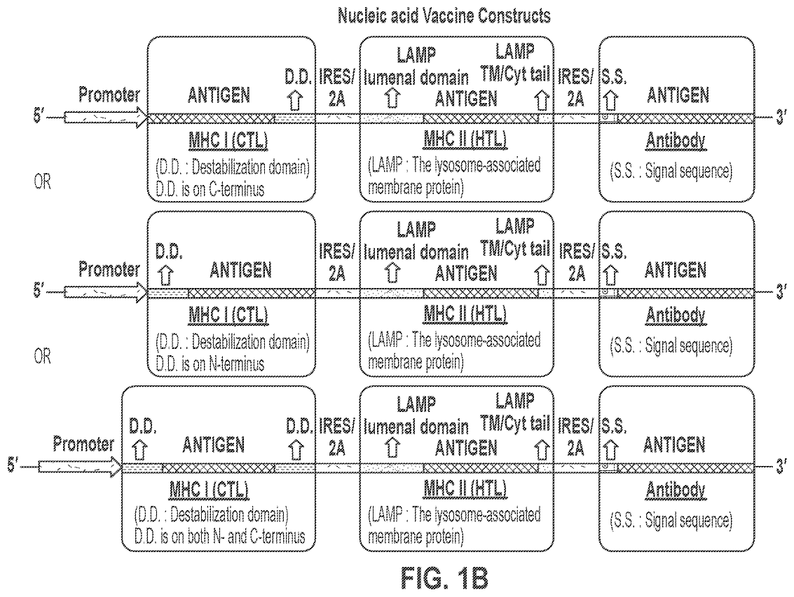

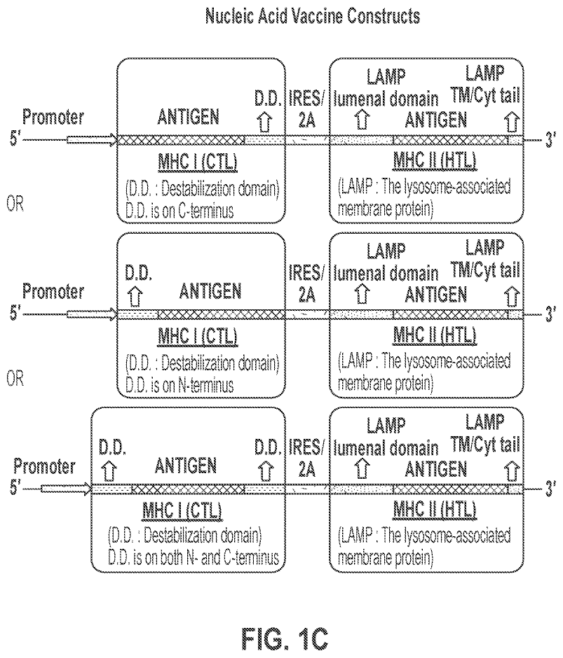

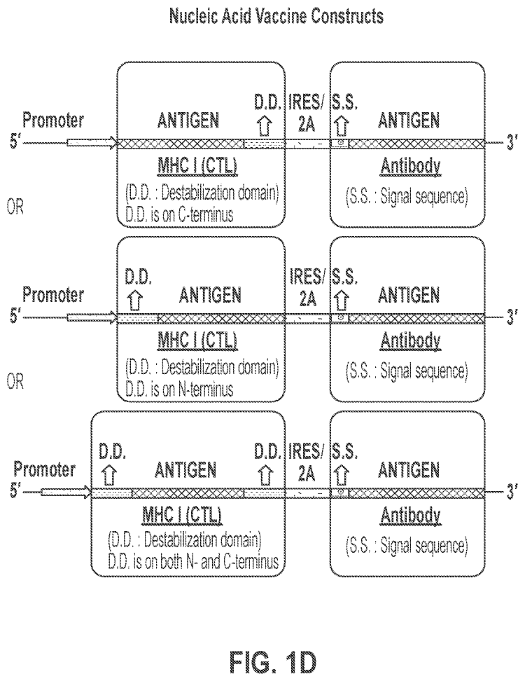

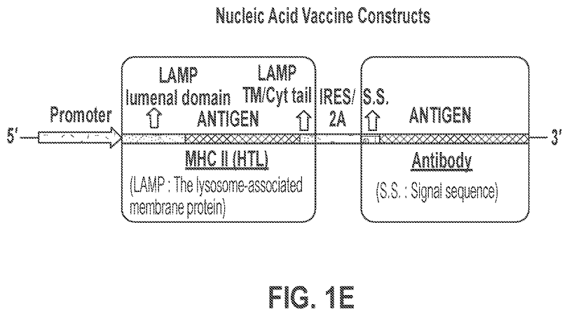

The polycistronic vaccine constructs encode at least one target antigen and comprise a plurality of independent cistrons, wherein each independent cistron encodes a modified target antigen, wherein the modified target antigen comprises an in-frame fusion protein of a target antigen and at least one specific domain that dictates the processing and presentation of the antigen. In certain embodiments, the domain comprises a destabilization domain (D.D.), a lysosome-associated membrane protein (LAMP) domain and a signal sequence (s.s.). In certain embodiments, the domain can comprise any combination (e.g., a fusion) of a D.D. domain, a LAMP domain, and a signal sequence (s.s.). The polycistronic vaccine constructs of the invention can comprise without limitation, any number of independent cistrons, for example, at least two independent cistrons, two independent cistrons, three independent cistrons, three independent cistrons, four independent cistrons, independent cistrons, six independent cistrons, seven independent cistrons, eight independent cistrons, nine independent cistrons, ten independent cistrons, eleven independent cistrons, twelve independent cistrons, thirteen independent cistrons, fourteen independent cistrons, fifteen independent cistrons, independent cistrons, sixteen independent cistrons, seventeen independent cistrons, eighteen independent cistrons, nineteen independent cistrons, twenty independent cistrons or greater than twenty independent cistrons. The specific domains comprised by the polycistronic vaccine constructs of the instant invention provide specific functional characteristics that contribute to enhancement of the immune response to the target antigen(s). The polycistronic vaccine constructs provided by the invention can comprise without limitation, modified target antigens comprising any number of the specific domains, including two or more of the same domain (e.g., two D.D. domains) within a single polycistronic construct. Exemplary polycistronic vaccine constructs provided by the invention are shown in FIGS. 1A-IE and 2A-2E, and illustrate the differences in the design of a DNA vaccine construct versus an mRNA vaccine construct. For example, polycistronic vaccine constructs for DNA vaccines comprise a suitable mammalian promoter to allow for the transcription of the encoded mRNA (FIGS. 1A-1E), whereas polycistronic vaccine constructs for mRNA vaccines comprise a coding region is flanked by sequences corresponding to a 5′ 7-methylguanosine triphosphate (m⁷G) cap and 5′ untranslated region (5′ UTR) including Kozak sequence at the 5′ end, and 3′ untranslated region (3′UTR) that includes a poly(A) tail, and optionally a 3′-immunoenhancing element (IE) at the 3′ end (FIGS. 2A-2E). The IE sequence can comprise two complementary single-stranded RNA separated by a small loop sequence. In certain embodiments, the IE sequence comprises a 3′-terminal double-stranded RNA spanning about 50-5000 base-pairs (bp). In certain embodiments, the IE sequence is about 50 bp, about 100 bp, about 200 bp, about 300 bp, about 400 bp, about 500 bp, about 1000 bp, about 2000 bp, about 3000 bp, about 4000 bp, up to about 5000 bp. The double-stranded RNA can comprise polyG:C or polyA:U. In certain embodiments, the double-stranded RNA is a random combination of A, U, G, C, which can be optimized to have no or little sequence similarity with any endogenous mammalian RNA sequences. The IE sequences likely stimulate dendritic cells as has been reported with poly T sequences.

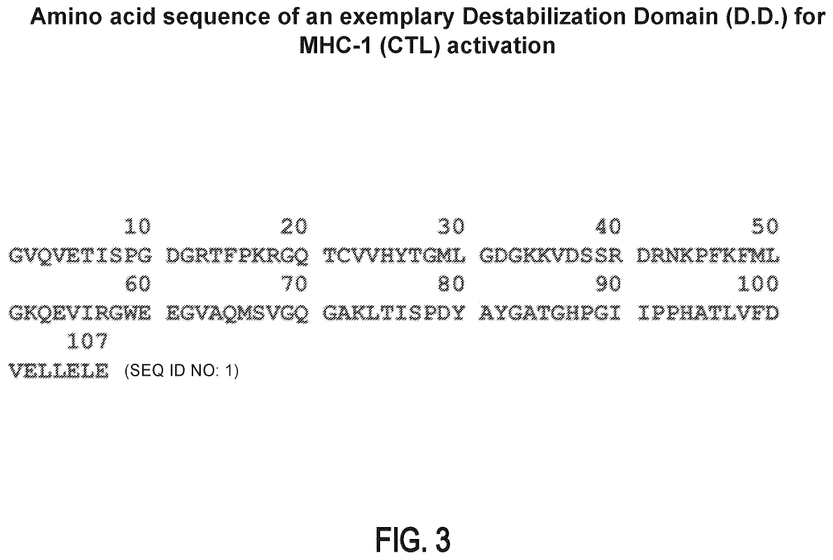

Destabilization Domain (D.D.) for MHC-1 (CTL) activation: In certain embodiments of the present invention, in the polycistronic vaccine constructs provided by the present invention, the selected (target) antigen or antigens is/are modified at either the amino (—N) or carboxyl (—C) terminus, by the addition of a protein destabilization domain, which typically is a 107 amino acid sequence that confers instability to the entire protein (to which it is fused), facilitating its rapid proteosomal degradation (Navarro, R. et al. (2016) ACS Chem Biol. August 19;11(8):2101-4) (FIG. 3). As such any mutation in D.D. that causes destabilization is within the scope of the present invention, and can be used in vaccine design. Methods for screening and/or identifying D.D. mutants of protein are described for example in Banaszynski et al., Cell, v126: 995-1004; U.S. Pat. Appl. Pub. No. 20090215169; and U.S. Pat. No. 8,173,792. Exemplary D.D. domains that are within the scope of the present invention can include, but are not limited to: D.D. sequences shown in FIG. 3, and in Examples 1 and 5; D.D. comprising a wild type or mutant human protein, bacterial protein or viral protein that undergoes proteasome-mediated degradation (human protein avoids undesirable immunogenicity); D.D. comprising a destabilizing sequence that is identified from a screening assay from a pool of mutants of any endogenous proteins; D.D. comprising a destabilizing mutant of human FKBP12, e.g., including but not limited to the F15S, V24A, L30P, E60G, M66T, R71G, D100N, E102G, K105I, E107G, L106P mutations, and any combination thereof; and D.D. is a derived from a known wild type protein that is turned over through proteasome degradation, e.g., including but not limited to cyclin A, C, D, and E; D.D. comprising IκB, which undergoes phosphorylation-dependent polyubiquitination and proteasome-mediated degradation upon activation by various surface signals including the toll-like receptor activation; D.D. comprising a wild type or mutant human protein, bacterial protein or viral protein that undergoes ligand-induced proteasome-mediated degradation. In certain embodiments, the wild type or mutant human protein, bacterial protein or viral protein that undergoes ligand-induced proteasome-mediated degradation is a known receptor for a small molecule ligand and the ligand is conjugated to a compound that interacts with a E3 ubiquitin ligase or an adaptor protein to induce proteasome-mediated degradation. In certain embodiments, the adaptor protein is cereblon and the compound to be conjugated to the ligand is thalidomide, pomalidomide or lenalidomide or a structurally related compound. In certain embodiments the E3 ubiquitin ligase is VHL and the compound to be conjugated to the ligand is a VHL-binding small molecule. Also within the scope of the instant invention is a D.D. domain having at least 50%, at least 60%, at least 70%, at least 80%, at least 85%, at least 90%, at least 95%, at least 96%, at least 97%, at least 98%, or at least 99% nucleic acid or amino acid sequence identity to any of the D.D. domain sequences described herein.

TABLE
Exemplary D.D. proteins

| DD protein | Genbank No. | Protein No. |
|---|---|---|
| FKBP12* | HGNC: 3711 | UniProtKB: P62942 |
| | Entrez Gene: 2280 | |
| | Ensembl: ENSG00000088832 | |
| Cyclin A | HGNC: 1578 | UniProtKB: P20248 |
| | Entrez Gene: 890 | |
| | Ensembl: ENSG00000145386 | |
| | OMIM: 123835 | |
| Cyclin B | HGNC: 1579 | UniProtKB: P14635 |
| | Entrez Gene: 891 | |
| | Ensembl: ENSG00000134057 | |
| | OMIM: 123836 | |
| Cyclin D1 | HGNC: 1582 | UniProtKB: P24385 |
| | Entrez Gene: 595 | |
| | Ensembl: ENSG00000110092 | |
| | OMIM: 168461 | |
| Cyclin D2 | HGNC: 1583 | UniProtKB: P30279 |
| | Entrez Gene: 894 | |
| | Ensembl: ENSG00000118971 | |
| | OMIM: 123833 | |
| Cyclin E1 | HGNC: 1589 | UniProtKB: P24864 |
| | Entrez Gene: 898 | |
| | Ensembl: ENSG00000105173 | |
| | OMIM: 123837 | |
| Cyclin E2 | HGNC: 1590 | UniProtKB: 096020 |
| | Entrez Gene: 9134 | |
| | Ensembl: ENSG00000175305 | |
| | OMIM: 603775 | |
| I-Kappa-B- | HGNC: 7797 | UniProtKB: P25963 |
| Alpha | Entrez Gene: 4792 | |
| | Ensembl: ENSG00000100906 | |
| | OMIM: 164008 | |
| I-Kappa-B- | HGNC: 7798 | UniProtKB: Q15653 |
| Beta | Entrez Gene: 4793 | |
| | Ensembl: ENSG00000104825 | |
| | OMIM: 604495 | |

*Note:
Wild-type (naturally-occurring) FKBP12 is not a D.D. A mutation is required in FKBP to convert it into a D.D.

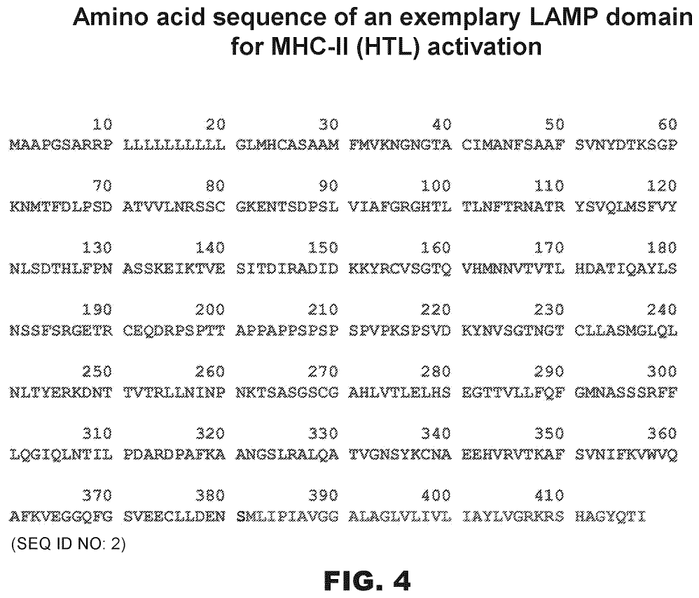

LAMP domain for MHC-II (HTL) activation: In certain embodiments of the present invention, in the polycistronic constructs provided by the present invention, the selected (target) antigen is modified by encoding the antigen sequence in the lumenal domain of the lysosome-associated membrane protein (LAMP) (FIG. 4) for trafficking to lysosomal compartments where it is co-localized with the MHC-II protein of professional antigen presenting cells for antigen presentation to helper T cells (HTL). The LAMP protein was first identified in the August laboratory (Chen, J. W. et al. (1985) J. Cell Biol. 101, 85-95; Chen, J. W. et al., (1986) Biochem. Soc. Symp. 51, 97-112; Guarnieri, F. G. et al. (1993) J. Biol. Chem. 268, 1941-1946; Raviprakash, K. et al. (2001) Virology 290, 74-82; Lu, Y. et al. (2003) Vaccine 21, 2187-2198; Anwar, A. et al. (2005) Virology 332:66-77; Arruda, L. B. et al. (2006) J. Immunol. 177: 2265-2275) (See also, U.S. Pat. No. 5,633,234, the content of which is incorporated by reference herein in its entirety). It was then shown that antigens encoded in DNA vaccines as a LAMP chimera elicited enhanced HTL and antibody responses (Wu, T-C. et al. (1995) Proc. Nat. Acad. Sci. USA 92, 11671-11675; Rowell, J. F. et al. (1995) J. Immunol. 155: 1818-1828; Ruff, A. L. et al. (1997) J. Biol. Chem. 272: 85671-8678; Lu, Y. et al. (2003) Vaccine, 21, 2187-2198; Marques, E. T. A. Jr. et al. (2003) J. Biol. Chem., 278: 37926-37936; deArruda, L. B. et al. (2004) Immunology 112:126-33; Chikhlikar, P. et al. (2006) PLoS One, 1:e135; Yang, K. et al. (2009) Gene Ther. 16 (11): 1353-62; Godinho, R. M. et al. (2014) PLoS One. June 16;9(6); Macile M. Jr. et al. (2015) PLoS Negl Trop Dis. 13;9(4):e0003693. doi: 10.1371/journal.pntd.0003693. eCollection April 13). As used herein a “LAMP domain” refers to a polynucleotide sequence or a polypeptide sequence encoding LAMP-1, LAMP-2, CD63/LAMP-3, DC-LAMP, or any lysosomal associated membrane protein, or homologs, orthologs, variants (e.g., allelic variants) and modified forms (e.g., comprising one or more mutations, either naturally occurring or engineered). In certain embodiments, a LAMP polypeptide is a mammalian lysosomal associated membrane protein, e.g., such as a human or mouse lysosomal associated membrane protein. More generally, a “lysosomal membrane protein” refers to any protein comprising a domain found in the membrane of an endosomal/lysosomal compartment or lysosome-related organelle and which further comprises a lumenal domain.

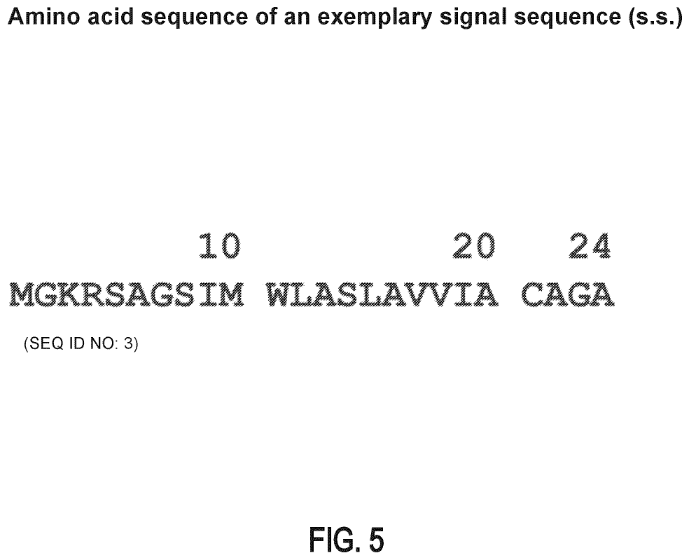

Antibody: In certain embodiments of the present invention, in the polycistronic constructs provided by the present invention, the selected antigen is modified by the addition of a signal sequence (s.s.), which is typically about 16-30 amino acids (aa) in length, at the N-terminus of newly synthesized protein that directs the antigen sequence to a secretory pathway for enhanced antibody activation. The function and use of signal sequences for vaccine applications has been widely reported (Davis, B. S., et al. (2001) J Virol. May; 75(9):4040-7. (FIG. 5). An exemplary s.s. provided by the instant invention comprises the amino acid sequence MGKRSAGSIM WLASLAVVIA CAGA (SEQ ID NO: 3) (FIG. 5). Variations, substitutions or modifications of the above sequence that retain the ability to direct the antigen sequence to a secretory pathway for enhanced antibody activation are within the scope of the instant invention. In certain embodiments, the length of the s.s. is about 16 aa, about 17 aa, about 18 aa, about 19, aa, about 20 aa, about 21 aa, about 22 aa, about 23 aa, about 24 aa, about 25 aa, about 26 aa, about 27 aa, about 28 aa, about 29aa, or about 30 aa. Also within the scope of the instant invention are s.s. which have a length less than 16 aa or greater than 30 aa, so long as they have the ability to direct the antigen sequence to a secretory pathway for enhanced antibody activation.

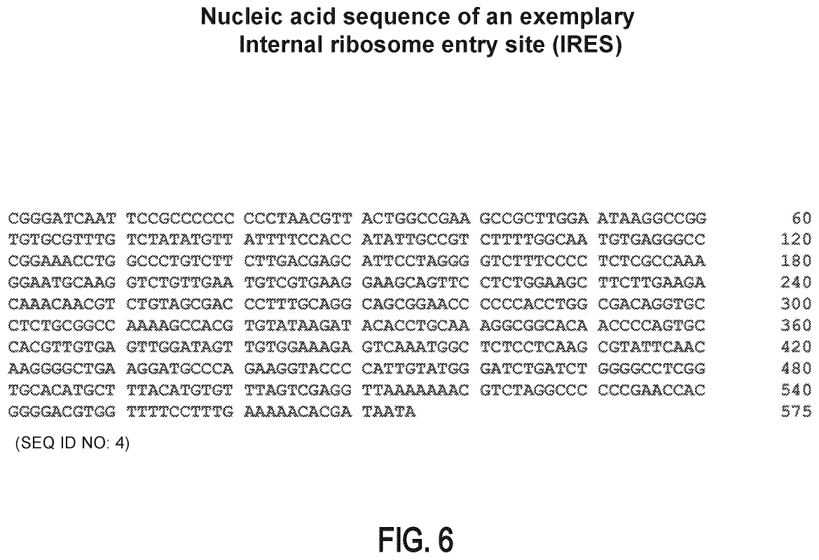

Internal ribosome entry sites (IRES): In certain embodiments of the present invention, in the polycistronic constructs provided by the present invention, each of the modified antigen sequences is translated as an independent cistron by the addition of internal ribosome entry sites (IRES) that mediate internal initiation of translation when present between the genes of interest (Holst, J. et al. (2006) Nat Protoc. 1(1):406-17). The IRES sequence thus allows the design of multicistronic expression cassettes to drive translation of several genes coded by the same mRNA with stable transgene expression and a constant ratio of the proteins of interest, in contrast to the use of different plasmids expressing each transgene An exemplary IRES sequence useful in the polycistronic constructs provided by the invention, includes, but is not limited to a nucleic acid sequence derived from an encephalomyocarditis virus (FIG. 6).

In certain embodiments, each of the independent cistrons of the polycistronic constructs are operably linked by an in-frame 2A self-cleaving peptide-based cleavage site. Exemplary 2A self-cleaving peptide-based cleavage site sequences include, but are not limited to P2A (porcine teschovirus-1 2A), T2A (Thoseaasigna virus 2A), E2A (equine rhinitis A virus (ERAV) 2A), F2A (FMDV 2A). See, table below.

TABLE
Sequences of exemplary self-cleaving peptides

| | |
|---|---|
| T2A | (GSG)EGRGSLLTCGDVEENPG↓P (SEQ ID NO: 5) |
| P2A | (GSG)ATNFSLLKQAGDVEENPG↓P (SEQ ID NO: 6) |
| E2A | (GSG)QCTNYALLKLAGDVESNPG↓P (SEQ ID NO: 7) |
| F2A | (GSG)VKQTLNFDLLKLAGDVESNPG↓P (SEQ ID NO: 8) |

*The ″cleavage″ occurs between the Glycine and Proline residues found on the C-terminus
**(GSG) residues can be added to the 5′ end of the peptide to improve cleavage efficiency

In certain embodiments, the polycistronic vaccine constructs provided by the invention comprise different, distinct combinations of independent cistrons (e.g., two independent cistrons or three independent cistrons). For example, a polycistronic vaccine construct provided by the invention can comprise two independent cistrons, wherein a first cistron encodes a modified antigen encoding a target antigen fused to a D.D. domain (fused at either the N-terminus or C-terminus of the target antigen) and a second cistron encodes a modified target antigen fused to an LAMP domain, or a second cistron encodes a modified antigen fused to a signal sequence (s.s.) (fused at either the N-terminus or C-terminus of the target antigen). Exemplary polycistronic constructs illustrating distinct combinations of independent cistrons are shown in FIGS. 1A-IE (for polycistronic DNA vaccine constructs) and in FIGS. 2A-2E for mRNA vaccine constructs.

### Host Cells

Nucleic acid polycistronic vaccine constructs according to the invention can be expressed in a variety of host cells, including, but not limited to: prokaryotic cells (e.g., E. coli, Staphylococcus sp., Bacillus sp.); yeast cells (e.g., Saccharomyces sp.); insect cells; nematode cells; plant cells; amphibian cells (e.g., Xenopus); avian cells; and mammalian cells (e.g., human cells, mouse cells, mammalian cell lines, primary cultured mammalian cells, such as from dissected tissues). The nucleic acid polycistronic vaccine constructs can be introduced into the cell using any art recognized method, including but not limited to viral mediated gene transfer, liposome mediated transfer, transformation, transfection and transduction, e.g., viral-mediated gene transfer such as the use of vectors based on DNA viruses such as adenovirus, adeno-associated virus and herpes virus, as well as retroviral based vectors.

The nucleic acid polycistronic vaccine constructs can be expressed in host cells isolated from an organism, host cells which are part of an organism, or host cells which are introduced into an organism. In certain embodiments, expression is in host cells in vitro, e.g., in culture. In certain embodiments, are expressed in a transgenic organism (e.g., a transgenic mouse, rat, rabbit, pig, primate, etc.) that comprises somatic and/or germline cells comprising any of the nucleic acids of the invention. Methods for constructing transgenic animals are well known in the art and are routine.

The nucleic acid polycistronic vaccine constructs also can be introduced in vitro, ex vivo or in vivo into cells, for example, stem cells, antigen presenting cells (APCs) such as dendritic cells, macrophages, monocytes, B-cells, artificially generated APCs, erythrocytes, gamma delta T lymphocytes, hematopoietic cells (myeloid cells, e.g., neutrophils, mast cells, eosinophils; and lymphoid cells), and endothelial cells, or can be introduced or administered directly into a host organism. As used herein, the term “antigen presenting cell” encompasses any cell which presents on its surface an antigen in association with a major histocompatibility complex molecule, or portion thereof, or, alternatively, one or more non-classical MHC molecules, or a portion thereof. Examples of suitable APCs include, but are not limited to, whole cells such as macrophages, monocytes, dendritic cells, B cells, artificially generated APCs, erythrocytes, gamma delta T lymphocytes, hybrid APCs, and foster antigen presenting cells. The cells may be heterologous or autologous with respect to the host organism. For example, cells can be obtained from the host organism, nucleic acid vectors introduced into the cells in vitro, and then reintroduced into the host organism.

In the context of APCs, “isolated” or “purified” population of cells is substantially free of cells and materials with which it is associated in nature. By substantially free or substantially purified APCs is meant at least 50% of the population are APCs, preferably at least 70%, more preferably at least 80%, and even more preferably at least 90% free of non-APCs cells with which they are associated in nature.

### Adaptive Immune Responses

As discussed above, the polycistronic vaccine constructs and the vaccine compositions comprising the same, provided by the present invention are capable of eliciting an enhanced activation of each of the three arms of the adaptive immune response: CD8+ cytolytic T lymphocyte (CTL), CD4+ helper T lymphocyte (HTL) and antibody, by virtue of the specific functionalities conferred by the LAMP, D.D., and s.s. domains, respectively. The particular design of the polycistronic constructs provided by the invention confers on them the ability to simultaneously activate all three arms of the adaptive immune, and therefore to advantageously enhance antigen-specific immune responses.

As used herein, “immune effector cells” refers to cells capable of binding an antigen and which mediate an immune response. These cells include, but are not limited to, T cells, B cells, monocytes, macrophages, NK cells and cytotoxic T lymphocytes (CTLs), for example CTL lines, CTL clones, and CTLs from tumor, inflammatory, or other infiltrates.

As used herein, an “enhanced adaptive immune response,” or “enhanced antigen-specific vaccination,” is defined as an increase in humoral and/or cellular responses to a specific target antigen encoded by the vaccine constructs provided by the present invention (chimeric/fusion constructs and polycistronic vaccine constructs) as ascertained qualitatively or quantitatively, for example, by an increase in the production of one or more immunological effectors such as cytokines (e.g., Interleukin-2 (IL-2), Perforin, Granzyme B, Interferon gamma (IFN-γ), Tumor necrosis factor alpha (TNF-α), Interleukin-4 (IL-4), Interleukin-5 (IL-5), Interleukin-6 (IL-6) and Interleukin-10 (IL-10)), or by an increase in the numbers of antigen-specific CD8⁺ cytolytic T lymphocytes (CTL), antigen-specific CD4⁺ helper T lymphocytes (HTL), and antigen-specific antibody production, or a combination thereof.

The adaptive immune system is one of the two main immunological strategies found in vertebrates (the other being the innate immune system). Adaptive immunity creates immunological memory after an initial response to a specific pathogen, and leads to an enhanced response to subsequent encounters with that pathogen. This process of acquired immunity is the basis of vaccination. Like the innate system, the adaptive system includes both humoral immunity components and cell-mediated immunity components. Unlike the innate immune system, the adaptive immune system is highly specific to a particular antigen (e.g., pathogen). For certain antigens, adaptive immunity can provide long-lasting protection.

Two major compartments constitute the adaptive immune system of mammals: the humoral and the cellular system. Humoral immunity is mediated by soluble protein molecules (antibodies) secreted by a specialized class of lymphocytes (B cells) into body fluids. The variable (polymorphic) part of the antibody molecules binds directly to native and denatured antigens (of diverse chemical composition) in the soluble phase. In contrast, cellular immunity is mediated by a different class of lymphocytes (T cells) that recognize only cell-associated protein antigens that have been intracellularly processed (partially digested) and are presented on the cell surface of antigen presenting cells (APC) in the context of major histocompatibility complex (MHC) glycoproteins.

Specific, protective immune reactivity is generated when the right type of effector function is delivered in sufficient strength at the right time at the site of emergence of ‘foreign’ antigen in the organism. Antigen-recognizing T lymphocytes potentially express a large repertoire of effector molecules, such as for example cytokines. A particular antigen-stimulated clone expresses only a minor subset of effector molecules from the large potential repertoire, i.e., only a limited set of effector functions is co-expressed in a particular T lymphocyte clone while most effector molecules are not produced. Particular functional phenotypes of T cells are hence clonally distributed. This known finding is of importance for vaccine designs because the preparation of an antigen and its mode of delivery critically influence the type of immune response it elicits. Depending on the mode of vaccination, the natural challenge of a vaccinated host with the respective pathogen may lead, either to stable protection, or to aggravation of disease and immunopathology.

The cellular immune system: The T cell system is composed of two subsets that differ in surface marker expression and functional phenotype, as well as in the restricting class of MHC molecules that present antigenic peptides to the respective T cell subsets. CD8⁺ killer (cytotoxic) T lymphocytes (CTL) recognize antigen in the context of MHC class I molecules, are often cytotoxic, and express interferon γ. CD4⁺ helper T cells (HTL) recognize antigen in the context of MHC class II molecules express different profiles of cytokines and are important in helping CTL activity and antibody production. Class I, cytotoxic T cell responses, occur in all nucleated cells and are the result of MHC class I protein binding the proteosomal fragments of cellular protein and presentation of these sequences to cytotoxic T cells; MHC class II proteins are present in professional antigen presenting cells (dendritic cells, macrophages, phagocytes, B-cells) and present the proteosomal fragments of proteins in these cells to the CD4⁺ helper T cells.

Membrane glycoproteins encoded within the MHC control specific activation of T cells. T cells do not recognize native antigens but respond to peptide fragments of protein antigens presented on the surface of antigen presenting cells (APC) by polymorphic MHC molecules. Processing of protein antigens is required to specifically stimulate T cells. Different pathways of intracellular processing of protein antigens control the activation of CD4⁺ and CD8⁺ T cell responses. In the exogenous processing pathway, extracellular protein antigens are endocytosed by APC and partially degraded in a specialized endosomal compartment to 12 to 15 residue peptides by acid proteolysis. These peptides bind haplotype-specific to MHC class II molecules and subsequently transit to the surface of the APC. Soluble protein antigens processed in this pathway preferentially elicit MHC class II-restricted CD4⁺ T cell responses. MHC class I-restricted CD8⁺ T cell responses are stimulated by protein antigens processed in the alternate endogenous processing pathway. In this pathway, antigenic peptides derived from cytosolic proteins are transported into the lumen of the endoplasmic reticulum (ER) by a peptide transporter complex where they bind to nascent MHC class I heavy chain/β₂m microglobulin dimers. This generates trimeric, transport-competent MHC class I complexes that move rapidly by the default secretory route to the APC surface. Thus, protein antigens derived from, either an exogenous, or an endogenous source, are processed in two alternative pathways for MHC restricted presentation of antigenic peptides to T cells.

### Delivery and Administration

In certain embodiments, the nucleic acid vaccine constructs of the invention can be formulated into a vaccine composition. As used herein, the term “vaccine composition” encompasses compositions comprising any of the polycistronic vaccine constructs (DNA, RNA, protein peptide) provided herein encoding at least one target antigen to which an immune response is desired, wherein the construct comprises a plurality of independent cistrons operably linked to a single promoter (in the 5′ to 3′ orientation), wherein each independent cistron encodes a modified target antigen comprising an in-frame fusion protein of the target antigen and at least one specific domain that dictates the processing and presentation of the antigen. The vaccine compositions can optionally comprise a pharmaceutically acceptable carrier useful for inducing an immune response in a host (subject). In some embodiments, the vaccines and vaccine compositions of the present invention are provided as “multivalent vaccine”. Herein, the term “multivalent” refers to a vaccine construct encoding two or more distinct antigens or modified antigens (e.g., comprising two or more different polynucleotide or polypeptides from different sources, e.g., fusion of two different tumor antigens or pathogen-derived antigens) or to a vaccine composition encompassing two or more distinct polycistronic constructs of the invention, which are co-administered as a mixture. The multivalent vaccine constructs can be administered by any of the methods or delivery routes described herein, including delivery via a nanoparticle system. In certain embodiments where the vaccine composition is in the form of an RNA vaccine, the RNA vaccine is produced by in vitro transcription of the DNA vector followed by 5′-capping of the RNA. In certain embodiments where the vaccine composition is in the form of an RNA vaccine, the RNA is made from chemically modified nucleotide blocks to enhance stability and cellular uptake in vivo. In certain embodiments, the DNA or RNA vaccine compositions of the instant invention may encode multiple different DNA or RNA antigens and may be co-administered as a mixture. As used herein, the terms “pharmaceutically acceptable carrier” and “pharmaceutically acceptable vehicle” are interchangeable and refer to a vehicle (e.g., fluid, lipid, or particle, viral and bacterial vectors) for containing vaccine antigens that can be introduced into a host without adverse effects. Suitable pharmaceutically acceptable carriers known in the art include, but are not limited to, sterile water, saline, glucose, dextrose, or buffered solutions, viral and bacterial vectors. Carriers may include auxiliary agents including, but not limited to, diluents, stabilizers (e.g., sugars and amino acids), preservatives, wetting agents, emulsifying agents, pH buffering agents, viscosity enhancing additives, colors, etc. Standard pharmaceutical texts, such as “Remington's Pharmaceutical Sciences,” 1990 may be consulted to prepare suitable preparations, without undue experimentation. The vaccine compositions provided by the invention can be administered in dosages and by techniques well known to those skilled in the medical or veterinary arts, taking into consideration such factors as the age, sex, weight, species and condition of the recipient animal, and the route of administration. The vaccine compositions of the invention can be administered by various routes, including but not limited to, subcutaneous, intramuscular, intravenous, intranasal or intradermal administration.

The amount of expressible DNA or transcribed RNA to be introduced into a vaccine recipient may vary depending on the strength of the transcriptional and translational promoters used. In addition, the magnitude of the immune response may depend on the level of protein expression and on the immunogenicity of the expressed gene product. In general, an effective dose ranges of about 1 ng to 5 mg, 100 ng to 2.5 mg, 1 μg to 750 μg, and about 10 μg to 300 μg of DNA is administered directly into a bodily tissue, such as muscle or dermal tissue. An exemplary dosage for intravenous administration of DNA is approximately 106-10²² copies of the DNA molecule. Subcutaneous injection, intradermal introduction, impression through the skin, and other modes of administration such as intraperitoneal, intravenous, or inhalation delivery are also suitable. For example, DNA is administered using a gene gun, e.g., gene gun particle-mediated DNA vaccination using a helium-driven gene gun. Booster vaccinations are administered in the same manner. Following vaccination with a nucleic acid vaccine, the immune response may also be boosted by administering a peptide or protein immunogen.

The nucleic acid can be administered naked, that is, unassociated with any proteins, adjuvants or other agents which affect the recipients' immune system. Naked DNA is administered in a physiologically acceptable solution, such as sterile saline or sterile buffered saline. Alternatively, the DNA may be associated with liposomes, such as lecithin liposomes or as a DNA-liposome mixture. Agents which assist in the cellular uptake of DNA (i.e., transfection facilitating agents), such as calcium ions may also be used. Microprojectiles coated with a polynucleotide are also useful as a means of administering the vaccine. In certain embodiments, the polycistronic vaccine constructs (DNA and RNA) described herein are formulated into and delivered via nanoparticles. Exemplary nanoparticles that are contemplated within the scope of the present invention include but are not limited to lipid nanoparticles (LNPs), and modified dendrimer nanoparticle (MDNP). Methods for preparing nanoparticles are known in the art and can be used to provide vaccine formulations in accordance with the instant invention. See, e.g., Oberli M. A. et al. (2017) Nano Lett. March 8;17(3):1326-1335; Chahal, J. S. et al. (2017) Sci Rep. March 21;7(1):252; Farris E. et al. (2016) Exp Biol Med (Maywood), May; 241(9):919-29. DNA vaccines provided by the instant infection can also be delivered to a subject using viral vectors (e.g., adenovirus, lentivirus, gamma-retrovirus) and bacterial vectors (e.g., Listeria monocytogenes, Salmonella typhimurium)

### RNA Vaccines

In certain embodiments, the present invention provides mRNA vaccines and mRNA based cellular vaccines. This includes direct delivery of mRNA vaccines into human subjects or transfecting mRNA into dendritic cells (DCs) B cells, neutrophils, PBMCs and any other cell populations.

In certain embodiments the mRNA vaccines provided by the present invention are prepared by in vitro transcription from any of the polycistronic vaccine constructs (FIG. 2A-2E) or any of the fusion constructs (FIGS. 14A-D) described herein. In certain embodiments, the RNA vaccine is produced by in vitro transcription of any of the polycistronic vaccine DNA (transcription) constructs followed by 5′-capping of the RNA. In certain embodiments, the mRNA vaccines can be made by total in vitro chemical synthesis (e.g., synthetic mRNA). In certain embodiments, the RNA vaccine is made from chemically modified nucleotide building blocks to enhance stability and cellular uptake in vivo.

Dendritic cell (DC) based-vaccination is an important approach to induce host antitumor immunity and has shown promising clinical efficacy in treating some tumors. Nevertheless, most clinical trials using DC vaccines in cancer therapy show only limited efficacy, suggesting a need for enhancing DC vaccine antigen presentation. Effective induction of antitumor T cell responses requires DC vaccines to efficiently present tumor associated antigens (TAA) and/or tumor specific antigens (TSA) including neoantigens. The polycistronic vaccine constructs provided by the present invention are designed to enhance DC antigen presentation to enhance both antitumor specific CD4⁺ and CD8⁺ T cell responses. DC vaccines transfected/infected with the above designed tumor antigen (TAA, TSA, neoantigens) expressing DNA, RNA, or viral and bacterial vectors allow for administration into patients by various routes, for example, intravenous (IV), intramuscular (IM), intradermal (ID), subcutaneous (S.C.), intratumoral or intranasal routes. This approach is not limited to specific target antigen, and is applicable to any antigen.

In certain embodiments, the mRNA vaccines provided by the present invention can be delivered either directly to patients or via the in vitro transfection/electroporation of mRNA vaccines into patient-derived dendritic cells (i.e., autologous dendritic cells) before reintroducing the transfected cells into patients. In certain embodiments, the mRNA vaccines will be elaborated into nanoparticles before administered into patients (IV or other routes). In addition, in both cases, the administration of vaccines can be further complemented by concurrent administration of an immunoadjuvant, for example, polyL:C, polyIC-LC, CpG, and other TLR ligands, particularly for mRNA nanoparticles, to further enhance antigen presentation.

In certain embodiments of the present invention, the dendritic cells source consists of their autologous precursors, e.g., peripheral blood mononuclear cell (PBMC)-derived monocytes. The monocytes are transdifferentiated into immature DCs upon 3-6 days culture in a growth medium (e.g., CellGro, AIM-V) supplemented with GM-CSF and IL-4. In certain embodiments of the present invention, the immature dendritic cells are matured upon or after loading.

In certain embodiments, the dendritic cells of the present invention are based on the expansion of autologous DCs from a human subject's peripheral blood. PBMCs are collected through leukapheresis followed by elutriation or gradient centrifugation (i.e., Ficoll gradient centrifugation), in order to increase the monocyte (Mo) fraction, which constitutes the selected DC precursors. This method of obtaining monocytes from individuals ensures both high purity and large amounts of DCs precursors that can be therefore cultured immediately, without the need of intermediate steps. The monocytes are thereafter differentiated into DCs in a GMP-conforming laboratory, with a culture medium, to render serum-free.

In certain embodiments, the present invention provides a method of manufacturing mRNA-loaded dendritic cells, the method comprising the steps of: (a) providing dendritic cells; and (b) transfecting the immature dendritic cells with one or more messenger RNA (mRNA) species transcribed in vitro from the polycistronic nucleic acid vaccine constructs provided by the invention. In some aspects, the present invention provides a method enhancing vaccine-induced T-lymphocyte responses comprising administering to a subject in need thereof, a composition comprising an isolated dendritic cell comprising one or more messenger RNA (mRNA) species transcribed in vitro from the polycistronic nucleic acid vaccine constructs provided by the invention. In other aspects, the present invention provides a method enhancing vaccine-induced T-lymphocyte responses comprising administering to a subject in need thereof, a composition comprising a first isolated dendritic cell and a second isolated dendritic cell each comprising one or more messenger RNA (mRNA) species transcribed in vitro from the polycistronic vaccine constructs or nucleic acid constructs provided by the invention. In particular aspects, the first and the second isolated dendritic cells each comprise different messenger RNA (mRNA) species or nucleic acid constructs provided by the invention. In certain embodiments, the mRNA encoding antigen can be delivered directly to patients. In certain embodiments, viral vectors (e.g., adenovirus, lentivirus, gamma-retrovirus) or bacterial vectors (e.g., Listeria monocytogenes, Salmonella typhimurium) incorporated with the DNA-encoding antigen expression cassette/construct can also be used to delivery antigen to dendritic cells or directly to patients.)

In certain embodiments of the present invention the DCs are loaded with the nucleic acid molecule (s) when they are still immature and later matured by means of adding one of the presently available standard cocktails (e.g., Ribomunyl, INF-γ, TNF-α, IL-1, PGE2, or combinations thereof). The mature DCs can be injected into the patients after sufficient antigens are loaded by different means including electroporation, liposome-mediated transfection, viral or bacterial mediated transduction etc. The antigen expression levels in the loaded DCs can be measured by methods known in the art such as RT-PCR, western blot analysis, and flow cytometry.

In certain embodiments, the present invention provides mRNA-based tumor vaccine or a dendritic cell vaccine comprising dendritic cells prepared according to the present invention. The mRNA-based vaccine or dendritic cell preparation may be employed in the treatment or prevention of virtually any type of cancer/tumor.

In certain embodiments of the present invention, the administration of DC preparation can be accompanied by the administration of immune-stimulatory agents and/or adjuvants. For example, the administration of vaccines can be further complemented by concurrent administration of an immunoadjuvant, including polyL:C, polyIC-LC and CpG, particularly for mRNA nanoparticles, to further enhance antigen presentation.

### Peptide and Protein Vaccines

In certain embodiments, peptides or polypeptides are expressed from any of the polycistronic vaccine constructs described herein, and can be used for any of the methods described herein. All herein designed vaccine constructs encoding infectious and pathologies antigens of, not limited to, e.g., Influenza A and B, HPV, HIV, will produce translation products in vivo as peptides and/or polypeptides. For the example of influenza, the vaccine is designed using the highly conserved HA sequence with full length M1, M2, and NS1 sequences as target antigen, the translation product is the polypeptide encoding these highly conserved HA, M1, M2, and NS1 sequences. The vaccines provided by the present invention can be delivered by any art-recognized delivery route, including but not limited to, oral, intramuscular (IM), intraperitoneal (IP), intravenous (IV) routes, or via electroporation.

### Methods

In some aspects, the present invention provides a method for modulating an immune response in a subject comprising administering any of the polycistronic vaccine constructs or vaccine compositions provided by the invention. In some aspects, the present invention provides a method for providing enhanced antigen-specific vaccination in a subject comprising administering any of the polycistronic vaccine constructs or vaccine compositions provided by the invention. In some aspects, the present invention provides a method for inducing a therapeutic immune response against a target antigen derived from a pathogen, a human self-protein or a malignant neoplasm comprising administration of any of the polycistronic vaccine constructs or vaccine compositions provided by the invention. In some aspects, the present invention provides a method for prevention and/or treatment of cancer in a subject in need thereof comprising administration of any of the polycistronic vaccine constructs, vaccine compositions, or dendritic cells (i.e., cellular therapy/cellular immunotherapy) provided by the invention. Additional methods provided by the present invention are set forth throughout the description.

By “subject” or “individual” or “patient” or “mammal,” which terms are used interchangeably herein, is meant any subject, particularly a mammalian subject, for whom diagnosis or therapy is desired. Mammalian subjects include for example, humans, domestic animals, farm animals, and zoo, sports, or pet animals such as dogs, cats, guinea pigs, rabbits, rats, mice, horses, cattle, and cows.

The terms “treat” or “treatment” refer to both therapeutic treatment and prophylactic or preventative measures, wherein the object is to prevent or slow down (lessen) an undesired physiological change or disorder, such as the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to or at risk of having the condition or disorder or those in which the condition or disorder is to be prevented. Any of these treatment types or types of patients may also be excluded.

As used herein, an “effective amount” is an amount sufficient to effect beneficial or desired results, e.g., such as an effective amount of nucleic acid transfer and/or expression, expression of a desired effector molecule(s) (e.g., cytokine), and/or the attainment of a desired therapeutic endpoint (e.g., partial or full reduction in size of a tumor). An effective amount can be administered in one or more administrations, applications or dosages. In one aspect, an effective amount of a polycistronic nucleic acid construct is an amount sufficient to transform/transduce/transfect at least one cell in a population of cells comprising at least two cells.

As used herein, a “therapeutically effective amount” is used to mean an amount sufficient to prevent, correct and/or normalize an abnormal physiological response or a measurable improvement in a desirable response (e.g., enhanced adaptive immune response). In one aspect, a “therapeutically effective amount” is an amount sufficient to reduce by at least about 30%, at least 50% at least 70%, at least 80%, or at least 90%, a clinically significant feature of pathology, such as for example, size of a tumor mass, antibody production, cytokine production, reduce pathogen (e.g., viral) load, fever or white cell count.

Herein, the terms “cancer,” “neoplasm,” and “tumor,” are used interchangeably and in either the singular or plural form, refer to cells that have undergone a malignant transformation that makes them pathological to the host organism. Primary cancer cells transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient.

Subjects who would benefit from the methods described herein include, but are not limited to a subject who is suffering from or is at risk of developing or suffering from a glioblastoma, bladder cancer, breast cancer, ovarian cancer, pancreatic cancer, and gastric cancer, cervical cancer, colon cancer, endometrial cancer, head and neck cancer, lung cancer, melanoma, multiple myeloma, leukemia, non-Hodgkin's lymphoma, prostate cancer, rectal cancer, malignant melanomas, alimentary/gastrointestinal tract cancer, liver cancer, skin cancer, lymphoma, kidney cancer, muscle cancer, bone cancer, brain cancer, eye or ocular cancer, rectal cancer, colon cancer, cervical cancer, bladder cancer, oral cancer, benign and malignant tumors, stomach cancer, corpus uteri, testicular cancer, renal cancer, throat cancer, acute lymphocytic leukemia, acute myelogenous leukemia, Ewing's Sarcoma, Kaposi's Sarcoma, basal cell carcinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, neurofibromatosis, tuberous sclerosis, hemangiomas, and lymphangiogenesis.

### EXAMPLES

The invention is now described with reference to the following Examples. These Examples are provided for the purpose of illustration only, and the invention is not limited to these Examples, but rather encompasses all variations which are evident as a result of the teachings provided herein.

### Example 1

### Nucleic Acid Polycistronic Vaccine Constructs Designed for Enhanced Activation of Adaptive Immune Responses to Pathogen and Malignant Neoplasm Antigens

Nucleic acid vaccine constructs were designed as depicted in FIG. 1A-1E for exemplary DNA vaccines or FIG. 2A-2E for exemplary mRNA vaccines (e.g., a TRIVAC™ vaccine).

Modified target antigens encoded by optimized codons and linked by the multicistrons element IRES and/or self-cleaving peptides 2A were integrated into the expression frames of DNA vaccine as illustrated in FIGS. 1A-1E and 2A-2E. For preparation of the Ovalbumin TriVac construct, the DNA sequences encoding Ovalbumin fusion with N-terminal mLAMP lumenal domain and transmembrane and cytoplasmic tail, C-terminal destabilization domain, IRES fragment, secreted ovalbumin domain, were chemically synthesized. All DNA fragments were ligated into p43 vector in the order of LAMP/OVA, IRES, DD/OVA, IRES, s.s.OVA via the MluI, BamHI, MunI, and NotI restriction sites. The p43 vector is described in: Yang et al., (2009) Gene Ther. November;16(11):1353-62, and Kessler, P. D. et al. (1996) Proc. Natl. Acad. Sci. USA; 93: 14082-14087. Sequences of mLAMP-OVA, D.D.-OVA, and s.s.- are shown below.

```
mLAMP-OVA (sequence of mLAMP lumenal and TM/cyt tail is underlined;
sequence of OVA is italicized)
(SEQ ID NO: 9)
                10 20 30 40 50 60
MAAPGARRPL LLLLLAGLAH GASALFEVKN NGTTCIMASF SASFLTTYET ANGSQIVNIS
                70 80 90 100 110 120
LPASAEVLKN GSSCGKENVS DPSLTITFGR GYLLTLNFTK NTTRYSVQHM YFTYNLSDTE
              130 140 150 160 170 180
HFPNAISKEI YTMDSTTDIK ADINKAYRCV SDIRVYMKNV TVVLRDATIQ AYLSSGNFSK
              190 200 210 220 230 240
EETHCTQDGP SPTTGPPSPS PPLVPTNPTV SKYNVTGNNG TCLLASMALQ LNITYLKKDN
              250 260 270 280 290 300
KTVTRAFNIS PNDTSSGSCG INLVTLKVEN KNRALELQFG MNASSSLFFL QGVRLNMTLP
              310 320 330 340 350 360
DALVPTFSIS NHSLKALQAT VGNSYKCNTE EHIFVSKMLS LNVFSVQVQA FKVDSDRFGS
              370 380 390 400 410 420
VEECVQDGNN VDMGSIGAAS MEFCFDVFKE LKVEHANENI FYCPIAIMSA LAMVYLGAKD
              430 440 450 460 470 480
STRTQINKVV RFDKLPGFGD SIEAQCGTSV NVHSSLRDIL NQITKPNDVY SFSLASRLYA
              490 500 510 520 530 540
EERYPILPEY LQCVKELYRG GLEPINFQTA ADQARELINS WVESQTNGII RNVLQPSSVD
              550 560 570 580 590 600
SQTAMVLVNA IVFKGLWEKA FKDEDTQAMP FRVTEQESKP VQMMYQIGLF RVASMASEKM
              610 620 630 640 650 660
KILELPFASG TMSMLVLLPD EVSGLEQLES IINFEKLTEW TSSNVMEERK IKVYLPRMKM
              670 680 690 700 710 720
EEKYNLTSVL MAMGITDVFS SSANLSGISS AESLKISQAV HAAHAEINEA GREVVGSAEA
              730 740 750 760 770 780
GVDAASVSEE FRADHPFLFC IKHIATNAVL FFGRCVSPTS MLIPIAVGGA LAGLVLIVLI
              790
AYLVGRKRSH AGYQTI
D.D.-OVA (sequence of D.D. is underlined; sequence of OVA is
italicized)
(SEQ ID NO: 10)
10 20 30 40 50 60
MGSIGAASME FCFDVFKELK VHHANENIFY CPIAIMSALA MVYLGAKDST RTQINKVVRF
                70 80 90 100 110 120
DKLPGFGDSI EAQCGTSVNV HSSLRDILNQ ITKPNDVYSF SLASRLYAEE RYPILPEYLQ
              130 140 150 160 170 180
CVKELYRGGL EPINFQTAAD QARELINSWV ESQTNGIIRN VLQPSSVDSQ TAMVLVNAIV
              190 200 210 220 230 240
FKGLWEKAFK DEDTQAMPFR VTEQESKPVQ MMYQIGLFRV ASMASEKMKI LELPFASGTM
              250 260 270 280 290 300
SMLVLLPDEV SGLEQLESII NFEKLTEWTS SNVMEERKIK VYLPRMKMEE KYNLTSVIMA
              310 320 330 340 350 360
MGITDVFSSS ANLSGISSAE SLKISQAVHA AHAEINEAGR EVVGSAEAGV DAASVSEEFR
              370 380 390 400 410 420
ADHPFLFCIK HIATNAVLFF GRCVSPEFGV QVETISPGDG RTFPKRGQTC VVHYTGMLGD
              430 440 450 460 470 480
GKKVDSSRDR NKPFKFMLGK QEVIRGWEEG VAQMSVGQGA KLTISPDYAY GATGHPGIIP
              490
PHATLVFDVE LLELE
s.s.-OVA (sequence of s.s. is underlined; sequence of OVA is
italicized)
(SEQ ID NO: 11)
                10 20 30 40 50 60
MGKRSAGSIM WLASLAVVIA CAGACTMGSI GAASMEFCFD VFKELKVHHA NENIFYCPIA
                70 80 90 100 110 120
IMSALAMVYL GAKDSTRTQI NKVVRFDKLP GFGDSIEAQC GTSVNVHSSL RDILNQITKP
              130 140 150 160 170 180
NDVYSFSLAS RLYAEERYPI LPEYLQCVKE LYRGGLEPIN FQTAADQARE LINSWVESQT
              190 200 210 220 230 240
NGIIRNVLQP SSVDSQTAMV LVNAIVFKGL WEKAFKDEDT QAMPFRVTEQ ESKPVQMMYQ
              250 260 270 280 290 300
IGLFRVASMA SEKMKILELP FASGTMSMLV LLPDEVSGLE QLESIINFEK LTEWTSSNVM
              310 320 330 340 350 360
EERKIKVYLP RMKMEEKYNI TSVIMAMGIT DVFSSSANLS GISSAESLKI SQAVHAAHAE
              370 380 390 400 410
INEAGREVVG SAEAGVDAAS VSEEFRADHP FLFCIKHIAT NAVLFFGRCV SP
```

### Example 2

Mouse Model Studies of TRIVAC™ Vaccine Constructs with Ovalbumin Antigen

Mouse immunization with the TRIVAC™ vaccine encoding the ovalbumin (OVA) antigen was used to compare the immune responses to the TRIVAC™ vaccine to other ovalbumin alone and other DNA vaccine constructs.

Immunization and splenocyte isolation and stimulation: Female Balb/c mice (Jackson Laboratory), 6-8 weeks old, 4 mice per group, were immunized intramuscularly (I.M.) twice, day 1 and day 15, with 50 μg, in 50 μl volume, of each construct as shown in FIGS. 1A-1E. On day 28, splenocytes from each mouse were isolated in culture medium (RPMI-1640 supplemented with 10% v/v fetal bovine serum, 100 U/ml penicillin/streptomycin, 2 mM L-glutamine, 50 μM 2-mercaptoethanol, and 0.01 M HEPES buffer). The single cell suspensions were depleted of red blood cells by ACK lysing buffer (Quality Biological), and were resuspended at 1×10⁷ cells ml-1 in the RPMI culture medium. Stimulation assays of the immunized mice immune responses were conducted with 1×10⁷ splenocytes per well that were cultured in a 12-well plate (Corning) with medium alone or with a final concentration of 20 ug/ml of ovalbumin protein. After incubation for 5 days at 37° C. in 5% CO², the culture supernatants were collected for detection of secreted cytokines as measured by ELISA kits (Invitrogen) following standard, recommended ELISA protocols.

Cytokine responses of mice immunized with the OVA, LAMP/OVA, D.D./OVA as single, mixed and IRES polycistronic constructs. Cytokine responses (IFN gamma, Granzyme B, IL-2, IL-10, IL-4, IL-5, and IL-6) were measured by ELISA assays with mice immunized by seven different vaccine formulations: (1) s.s.OVA (secreted OVA); (2) LAMP/OVA; (3) D.D./OVA; (4) Mixture of LAMP/OVA and D.D./OVA; (5) Mixture of LAMP/OVA, D.D./OVA and s.s.OVA; (6) polycistronic construct of LAMP/OVA-IRES-D.D./OVA; (7) Polycistronic construct of LAMP/OVA-IRES-D.D./OVA-IRES-s.s.OVA.

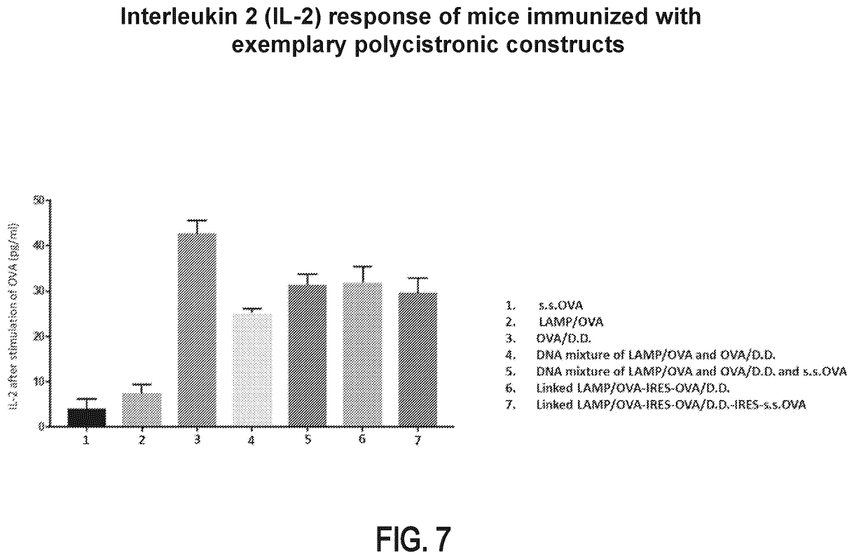

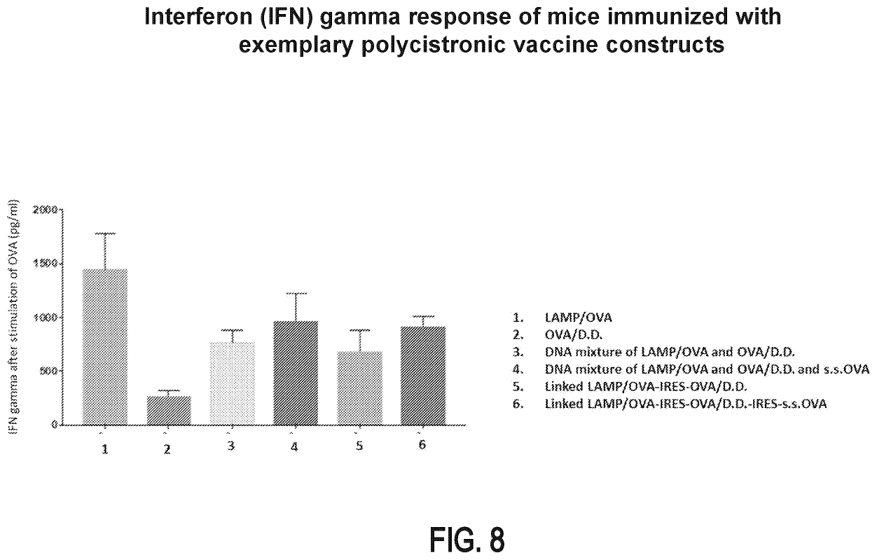

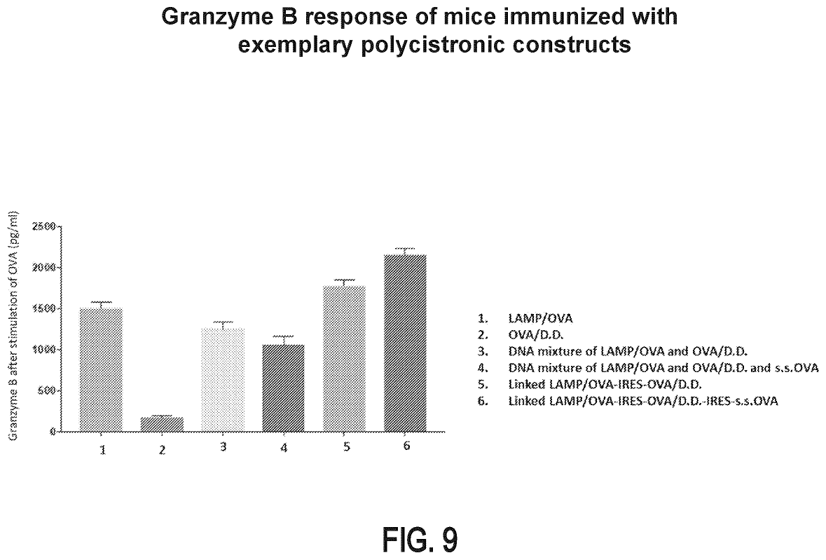

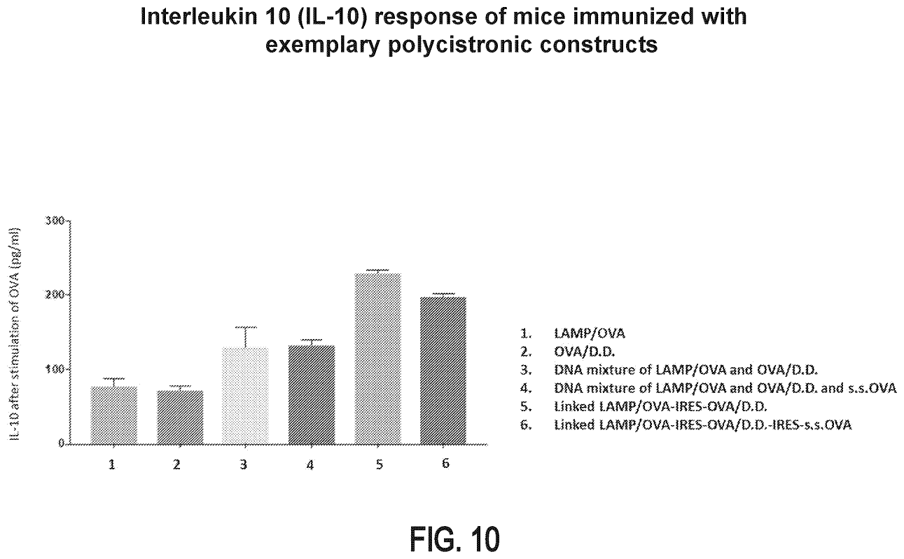

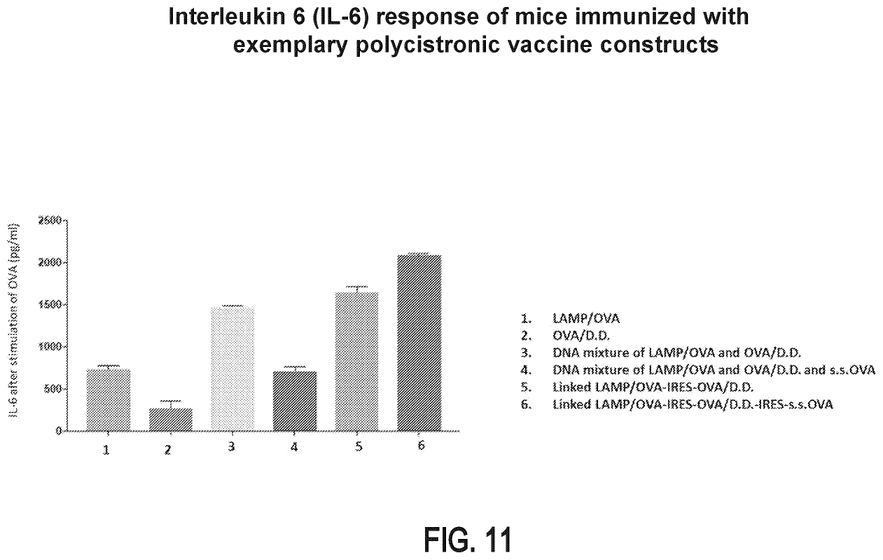

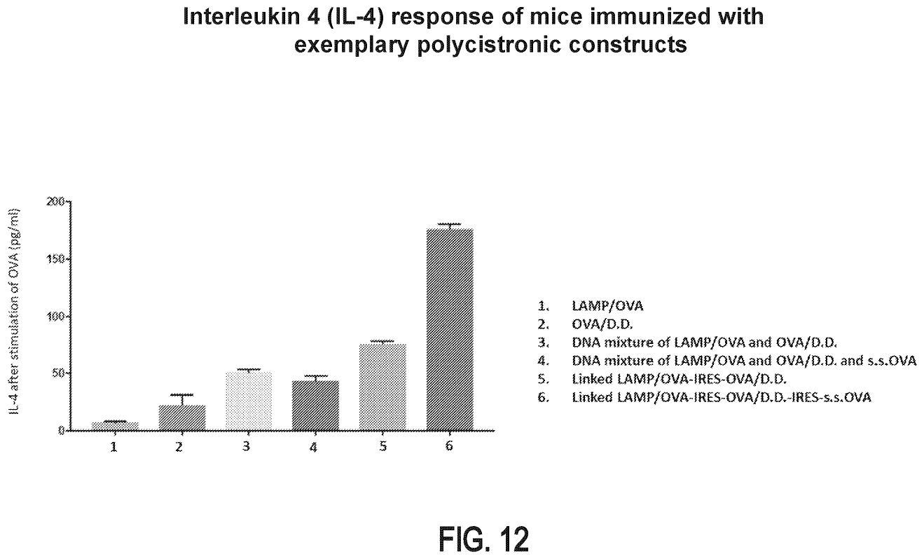

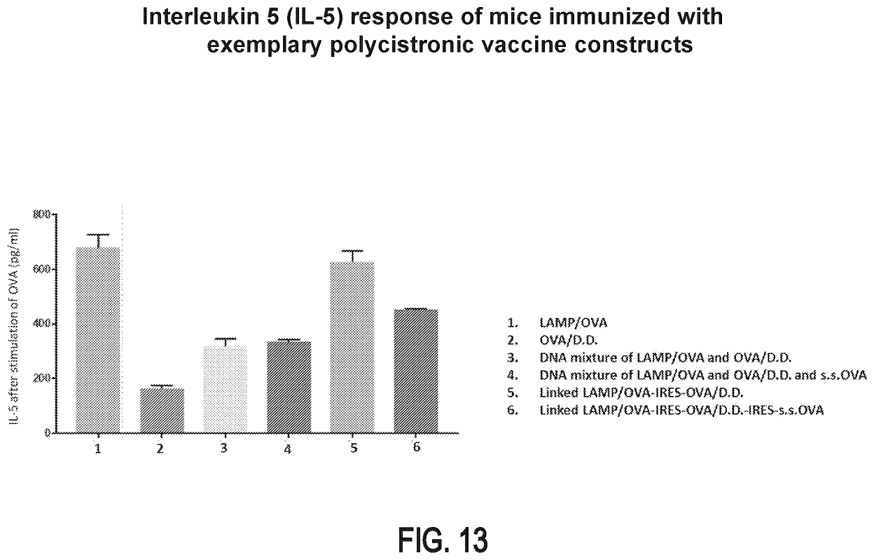

ELISA assay results showed high levels of production of all seven cytokines (IL-2, IFN gamma, Granzyme B, IL-10, IL-4, IL-5, and IL-6) with vaccines 6 and 7 (two linked construct groups) and vaccines 4 and 5 (two DNA mixture groups). LAMP/OVA trigged a robust production of IFN gamma as well as IL-5 (FIG. 8 and FIG. 13). D.D./OVA produced the highest level secretion of IL-2 (FIG. 7), and lower responses of other cytokines (IFN gamma, Granzyme B, IL-10, IL-4, IL-5, and IL-6). The highest response to a single construct (LAMP/OVA) was observed for IFN gamma and Granzyme B. Notably, the polycistronic construct of LAMP/OVA-IRES-D.D./OVA-IRES-s.s.OVA produced high responses of multiple cytokines (IL-2, IFN gamma, Granzyme B, IL-6, and IL-4).

Taken together, polycistronic constructs of LAMP/OVA-IRES-D.D./OVA-IRES-s.s.OVA resulted in marked broadening of helper T lymphocyte (HTL) and cytolytic T lymphocyte (CTL) responses, suggesting that the polycistronic constructs (CTL, HTL, and antibody) would be the most effective vaccine candidate for immunotherapy applications.

### Example 3

### Constructs to Enhance Human Dendritic Cell Vaccine Induced T Lymphocyte Responses

Dendritic cells (DCs) are the most potent professional antigen-presenting cells, capable of initiating adaptive immune responses by priming T lymphocytes. DC based-vaccination is an important approach to induce host anti-viral and anti-tumor immunity and has shown promising clinical efficacy in treating some tumors (De Vleeschouwer, S. et al. (2008) Clin Cancer Res. 14(10):3098-3104; Yu, J. S. et al. (2004) Cancer Res. 64(14):4973-4979; Cho, D. Y. et al. (2012) World Neurosurg. 77(5-6):736-744; Mitchell, D. A. et al. (2015) Nature. 519(7543):366-369; Bol, K. F. et al. (2015) Oncoimmunology. 4(8):e1019197; Jadidi-Niaragh, F. et al. (2017) J. Control. Release. 246:46-59). However, while antigen specific immune responses have been reported, the duration and magnitude of these responses are typically weak, and objective clinical responses have been limited (Elster, J. D. et al. (2016) Hum Vaccin Immunother. 12(9):2232-2239; Pajtasz-Piasecka, E. et al. (2010) Immunotherapy-UK 2(2):257-268; Kyte, J. A. et al. (2016) Oncoimmunology 5(11):e1232237. High antigen presentation efficiency is essential for an effective DC vaccine, so that it can induce strong T cell responses. D.D. domain and LAMP domain constructs linked with the cytomegalovirus pp65 antigen were designed. It was demonstrated that this novel vaccine construct can enhance DC antigen presentation and induce strong antigen specific T cells in vitro.

Constructs and mRNA Preparation: Plasmid pSP73-Sph/A64 was used as the mRNA template vector. The sequence encoding a selected antigen CMV pp65 with D.D. or/and LAMP was cloned into the plasmid pSP73-Sph/A64 (FIG. 14). In vitro transcription was performed with T7 RNA polymerase (Ambion) to generate mRNA. The transcribed mRNA was recovered after DNaseI (Ambion) digestion on RNeasy columns (Qiagen). mRNA quality was verified by agarose gel electrophoresis. mRNA concentration was measured spectrophotometrically and stored at −80° C. in small aliquots.

### Example 4

### In Vitro Studies of the Constructs to Enhance Human Dendritic Cell Vaccine Induced Antigen-Specific T Lymphocyte Responses.

### Materials and Methods:

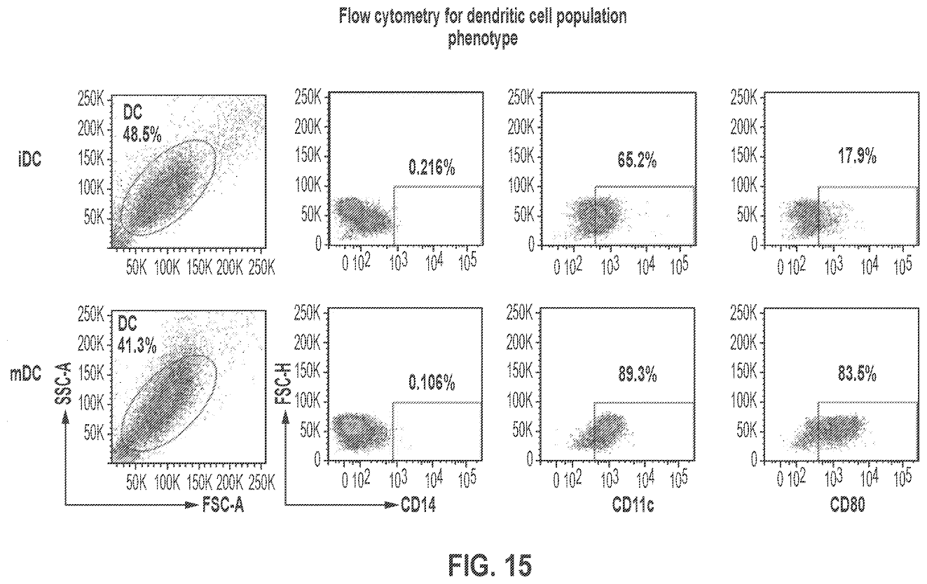

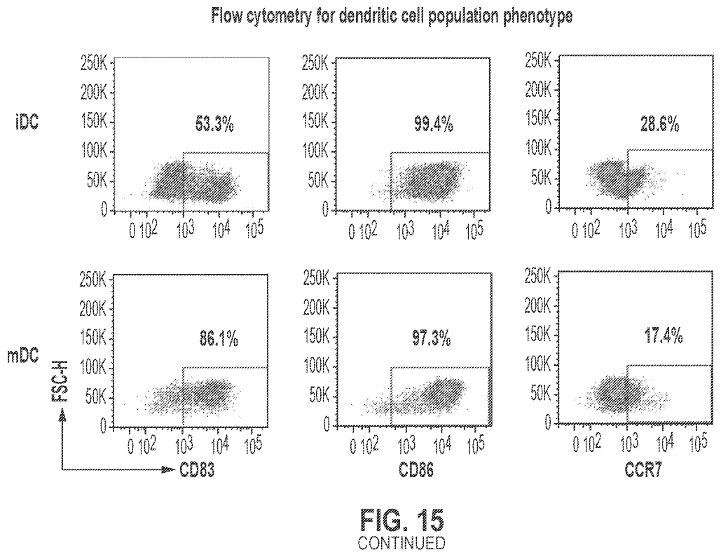

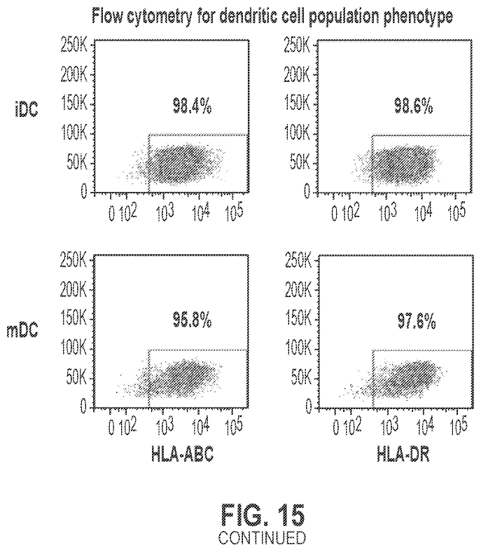

Dendritic Cells Preparation: Peripheral blood mononuclear cells (PBMCs) from healthy donors were cultured in 37° C. 5% CO₂ incubator for 2 hours. The adherent cells were then stimulated by 800 IU/ml GM-CSF and 500 IU/ml IL-4 in AIM-V media for 6 days to get immature dendritic cells (iDC). On day 6, 160 ng/ml IL6, 5 ng/ml TNF-α, 5 ng/ml IL-10 and 1 ug/ml PGE2 were added. On day 7, mature dendritic cells (mDC) were harvested. The phonotype of iDC and mDC was measured by flow cytometry (FIG. 15).

Electroporation of DCs with mRNAs: DCs were harvested and washed once with PBS and once with Opti-MEM without phenol red (Invitrogen Life Technologies). The cells were resuspended at a concentration of 5×10⁶/ml with Opti-MEM. RNA was transferred to a 4-mm cuvette. A volume of 200 μl of cell suspension was added and pulsed using an Electro Square Porator (ECM630, BTX, San Diego, CA). Pulse conditions were voltage, 300 V; capacitance, 150 μF; and resistance, 25Ω. 5 μg mRNA/10⁶ DCs were used for each electroporation. Immediately after electroporation, the cells were transferred to medium.

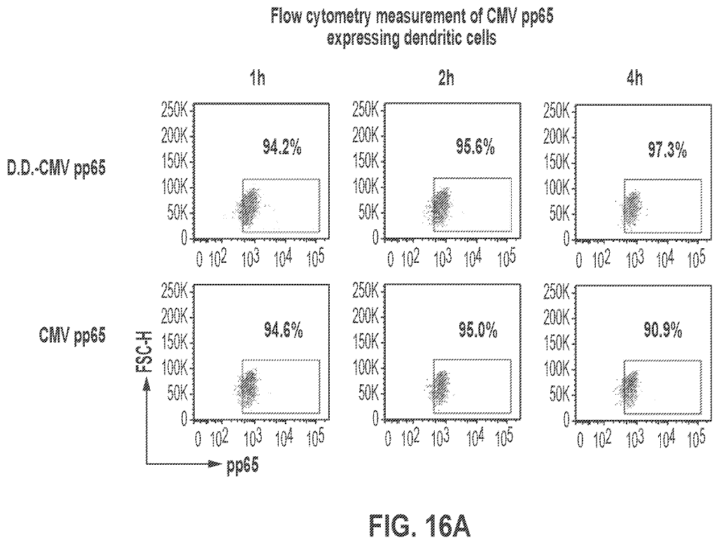

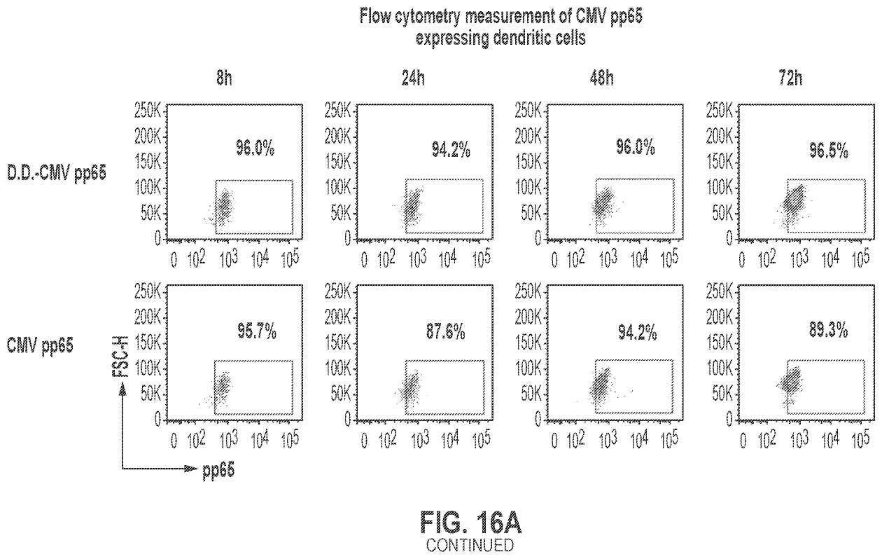

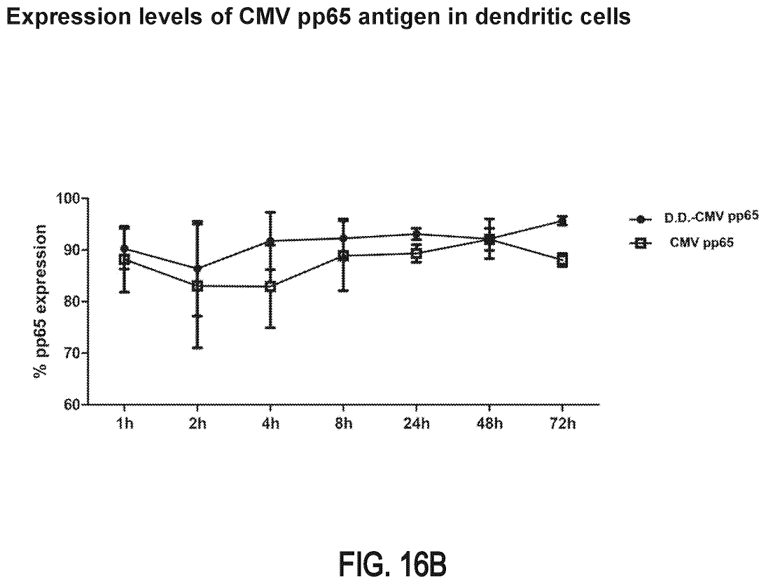

Expression Measurement of CMV pp65 Antigen in Dendritic Cells: D.D.-CMV pp65 mRNA and CMV pp65 mRNA were transfected into dendritic cells respectively by electroporation. Then, the expression levels of CMV pp65 antigen in dendritic cells were measured at 1 hour, 2 hours, 4 hours, 8 hours, 24 hours, 48 hours and 72 hours later by intracellular staining and then flow cytometry (FIG. 16).

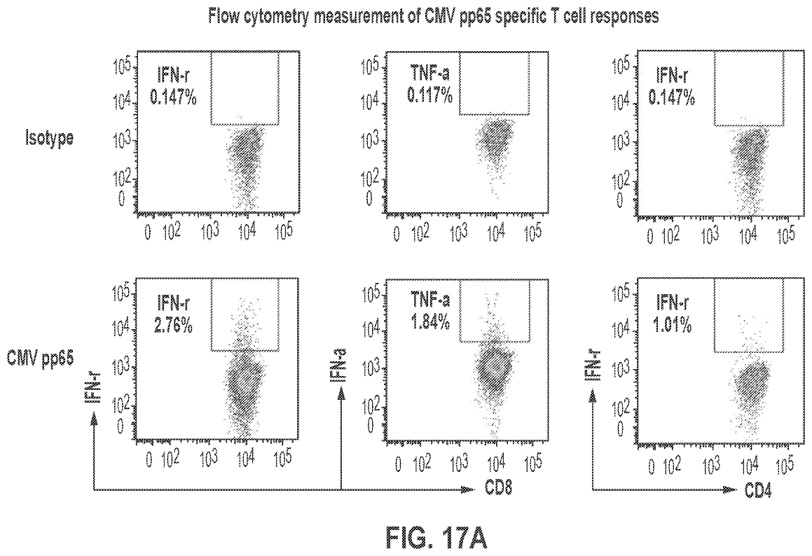

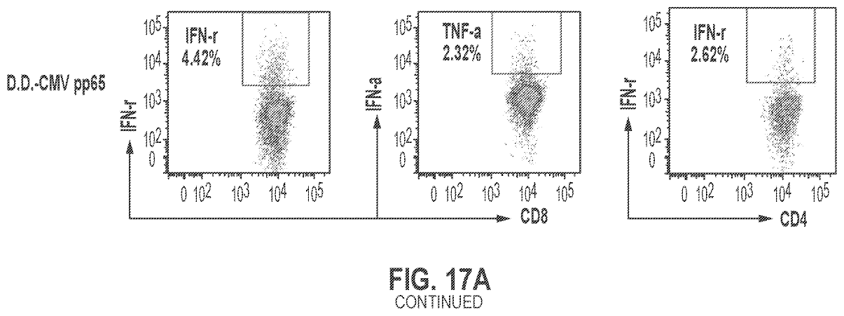

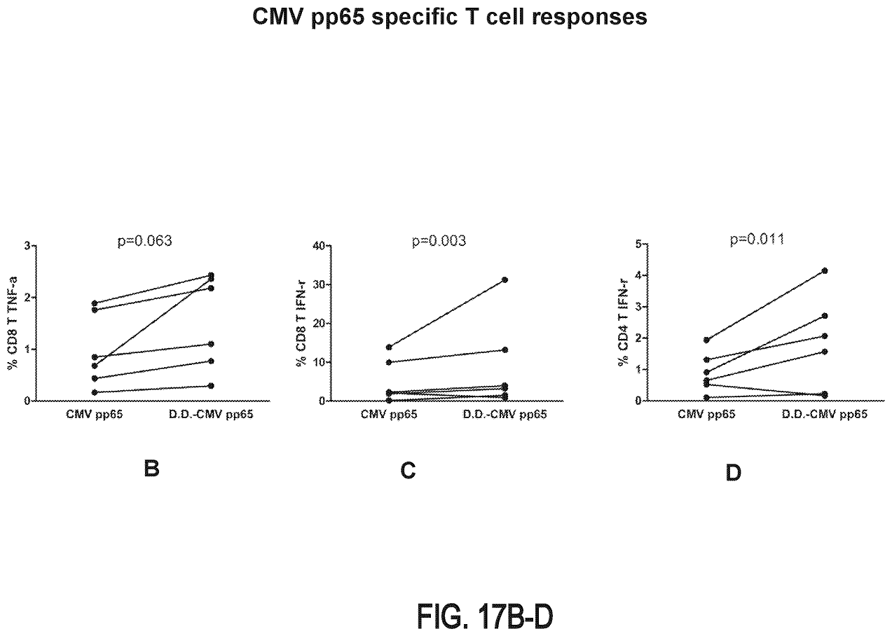

In Vitro Stimulation of T cells with mRNA Loaded-DCs: After electroporated with mRNAs, DCs were rested in DC culture medium at 37° C. for 4 hours. The mRNA-loaded DCs were then used to co-culture with PBMC at a rate of 1:10 in 37° C. 5% CO₂ incubator. On day 5, 50 U/ml IL-2 was added. On day 7, PBMCs were re-stimulated with mRNA loaded DCs. IL-2 and IL-7 were supplied every 3 days. On day 14, cells were harvested, CD8 T cell IFN-γ, TNF-α responses and CD4 T IFN-γ response were measured by flow cytometry (FIG. 17, 18, 19).

### Results:

D.D. attached antigen CMV pp65 was expressed stably and durably in DCs. DCs were prepared from PBMCs and the phonotype was measured by flow cytometry (FIG. 15). The CD11c⁺CD14⁻ population was gated as DCs. The costimulatory molecules (CD80, CD83, and CD86) and MHC molecules (HLA-ABC, HLA-DR) are essential in inducing T cells responses. The chemokine receptor CCR7 mediates DC migration towards T-cell areas in the lymph nodes. D.D.-CMV pp65 mRNA or CMV pp65 mRNA was then transfected into DCs respectively by electroporation and CMV pp65 expression was measured by intracellular staining with an anti-pp65 mAb (FIG. 16). The data showed that the antigen CMV pp65 was expressed stably and durably in DC within 72 hours with or without D.D attached, indicating that D.D. did not affect the protein expression levels of pp65 antigen.

D.D. enhanced human DC vaccine induced antigen-specific T cell responses. PBMCs from healthy donors were stimulated by D.D.-CMV pp65 mRNA or CMV pp65 mRNA loaded DC twice for 14 days. CD8+ T cell IFN-γ, TNF-α and CD4+ T cell IFN-γ responses were measured (FIG. 17). Compared with the CMV pp65 group, the D.D.-CMV pp65 group showed stronger CD8 T cell IFN-γ (p=0.003, Paired-Samples T Test was used, n=6), TNF-α (p=0.063) and CD4 T cell IFN-γ (p=0.011) responses.

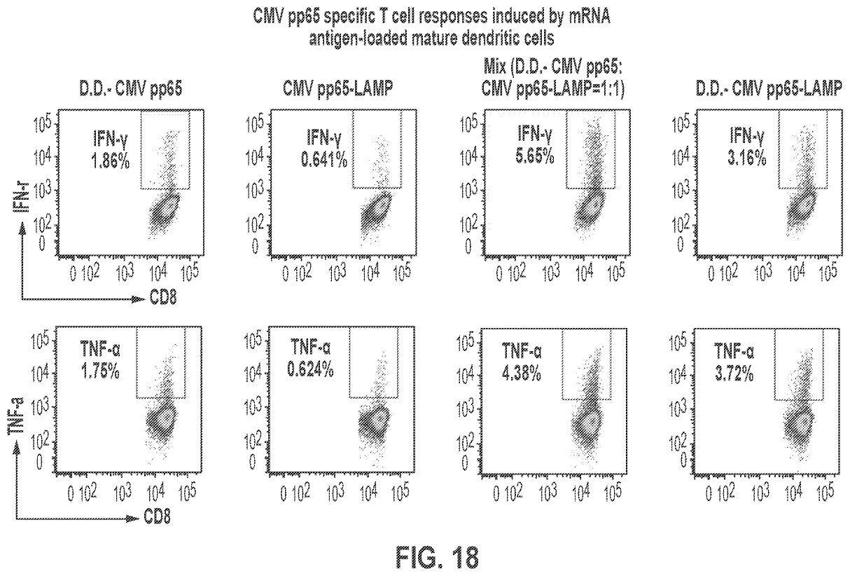

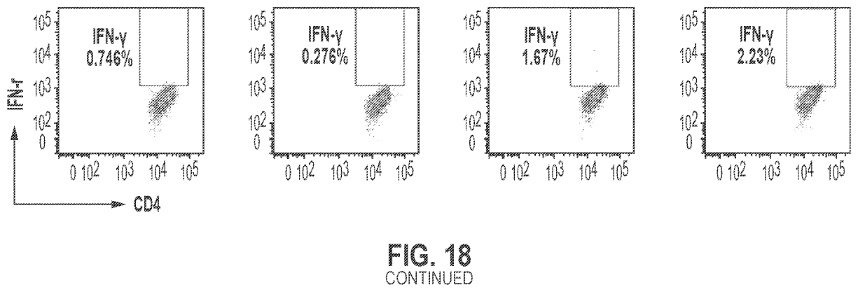

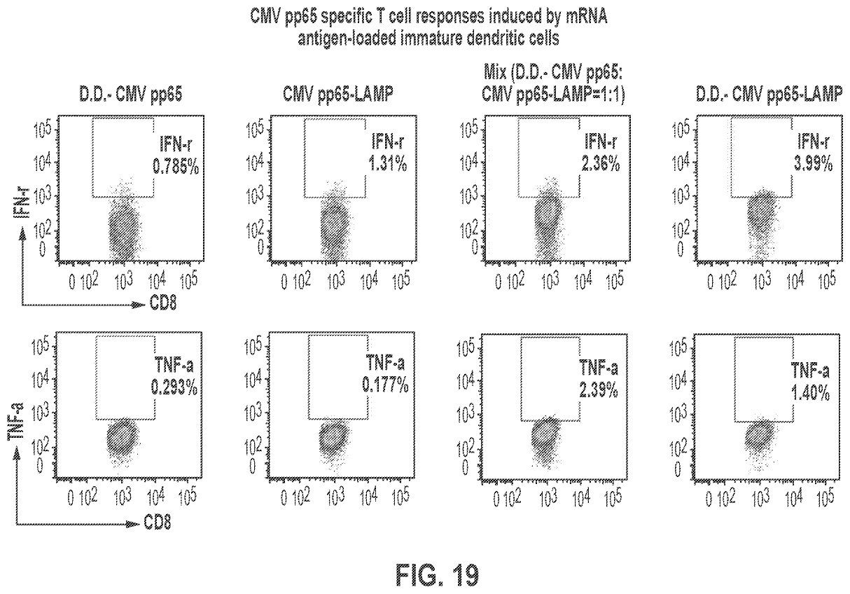

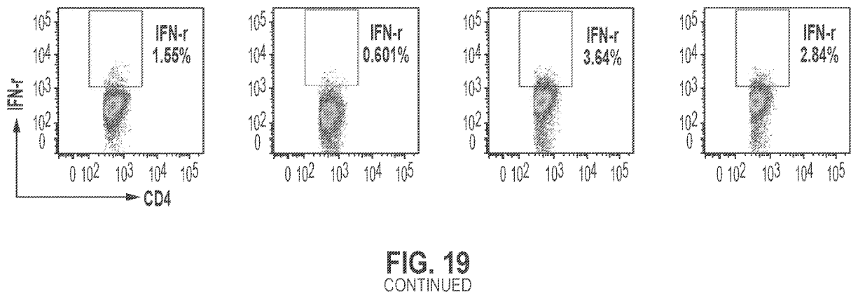

A further enhanced antigen-specific T cell response was observed when both D.D. domain and LAMP domain were included. Upon determination of D.D.-CMV pp65 induced stronger antigen-specific T cell responses, CMV pp65 fused to both D.D. domain and LAMP1 domain was generated as shown in FIG. 14. CMVpp65 fused to D.D or LAMP1 alone served as controls. The capability of the following constructs in inducing antigen-specific T cell responses was tested: 1. D.D.-CMVpp65; 2. CMVpp65-LAMP1; 3. Mix (D.D.-CMV pp65: CMV pp65-LAMP=1:1); 4. D.D.-CMV pp65-LAMP1 two domain fusion. The mRNAs for each group were transfected into mature dendritic cells (mDC, FIG. 18) or immature dendritic cells (iDC, FIG. 19) respectively by electroporation. Higher CD8+ effector T cells expressing IFN-γ, TNF-α and CD4+ effector T cell expressing IFN-γ in the Mix (D.D.-CMV pp65: CMV pp65-LAMP=1:1) and D.D.-CMV pp65-LAMP groups was observed than D.D.-CMV pp65 or CMV pp65-LAMP alone.

### Example 5

Tumor Associated Antigen Gp100 Specific T Cell Responses Induced by mRNA-Loaded DC

Constructs of gp100, LAMP-gp100, D.D.-gp100, s.s.-gp100 and LAMP-gp100-IRES-D.D.-gp100-IRES-s.s.-gp100 were generated using the methods described above. Then the DC vaccines were prepared and stimulated the autologous PBMC. Tumor associated antigen (TAA) gp100 specific T cell responses were measured by flow cytometry. Data showed that constructs with LAMP, D.D., s.s. or LAMP-D.D.-s.s. domains induced stronger T cell responses than gp100 control. This suggested that LAMP, D.D. and s.s. domains can enhance TAA-specific T cell priming in DC vaccine.

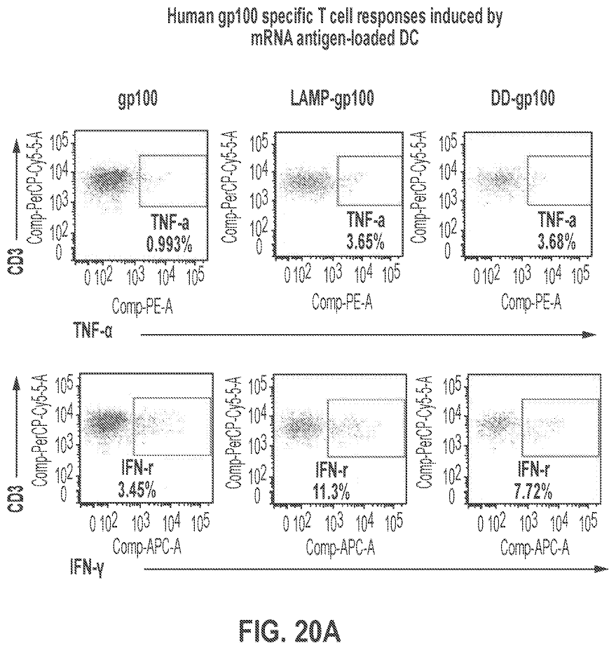

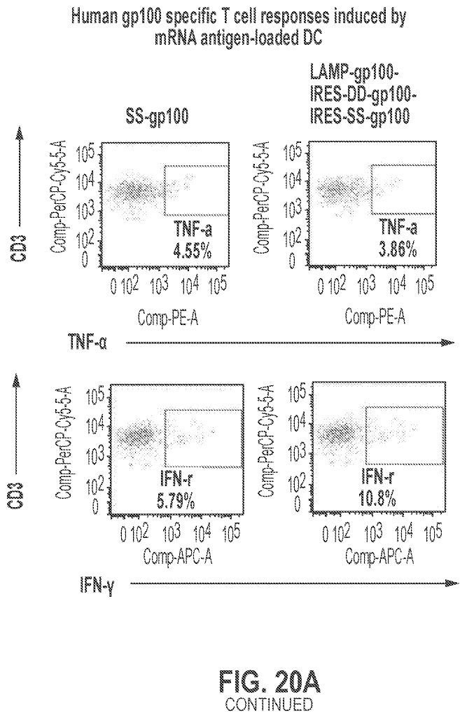

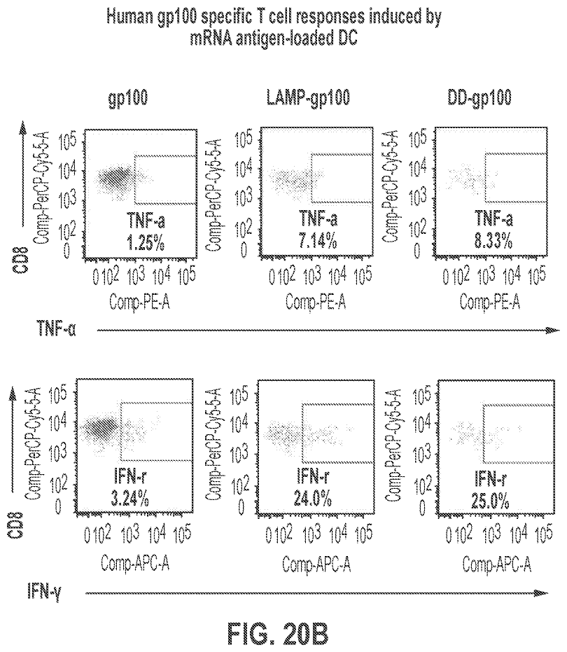

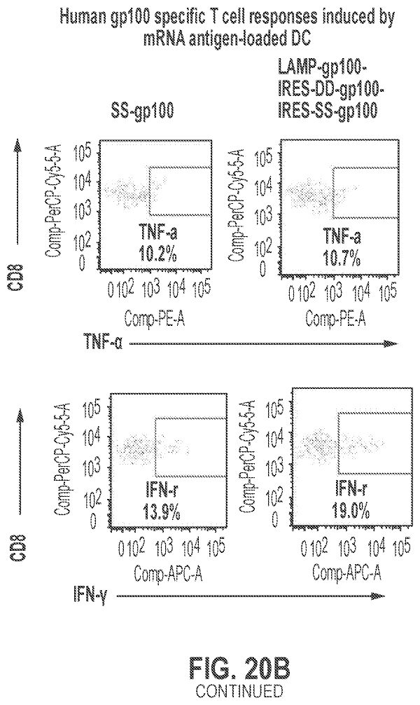

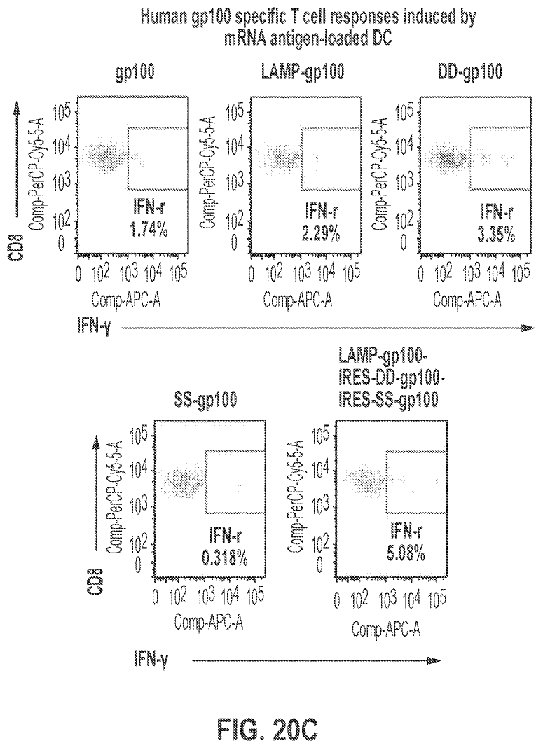

Human gp100, LAMP-gp100, D.D.-gp100, s.s.-gp100 or LAMP-gp100-IRES-D.D.-gp100-IRES-s.s.-gp100 mRNA was transfected into immature dendritic cells (iDC), respectively. The immature dendritic cells (iDC) were then further cultured to become mature dendritic cells (mDC). PBMCs from healthy donors were stimulated three times on day 0, day 7 and day 13 by mRNA loaded mDC above. During the cell culture process, 1p g/ml anti-human-PD-L and PD-L2 antibodies were added. CD3+ T cell TNF-α and IFN-γ, CD8 T cell TNF-α and IFN-γ and CD4 T cell IFN-γ responses were measured on day 14 by flow cytometry. (See, FIG. 20). The full length sequence of human gp 100, and sequences of hLAMP-hgp100, D.D.-hpg100, and s.s.-hgp100 are shown below.

```
Full length sequence of Human gp100
(SEQ ID NO: 12)
                10 20 30 40 50 60
MDLVLKRCLL HLAVIGALLA VGATKVPRNQ DWLGVSRQLR TKAWNRQLYP EWTEAQRLDC
                70 80 90 100 110 120
WRGGQVSLKV SNDGPTLIGA NASFSIALNF PGSQKVLPDG QVIWVNNTII NGSQVWGGQP
              130 140 150 160 170 180
VYPQETDDAC IFPDGGPCPS GSWSQKRSFV YVWKTWGQYW QVLGGPVSGL SIGTGRAMLG
              190 200 210 220 230 240
THTMEVTVYH RRGSRSYVPL AHSSSAFTIT DQVPFSVSVS QLRALDGGNK HFLRNQPLTF
              250 260 270 280 290 300
ALQLHDPSGY LAEADLSYTW DFGDSSGTLI SRALVVTHTY LEPGPVTAQV VLQAAIPLTS
              310 320 330 340 350 360
CGSSPVPGTT DGHRPTAEAP NTTAGQVPTT EVVGTTPGQA PTAEPSGTTS VQVPTTEVIS
              370 380 390 400 410 420
TAPVQMPTAE STGMTPEKVP VSEVMGTTLA EMSTPEATGM TPAEVSIVVL SGTTAAQVTT
              430 440 450 460 470 480
TEWVETTARE LPIPEPEGPD ASSIMSTESI TGSLGPLLDG TATLRLVKRQ VPLDCVLYRY
              490 500 510 520 530 540
GSFSVTLDIV QGIESAEILQ AVPSGEGDAF ELTVSCQGGL PKEACMEISS PGCQPPAQRL
              550 560 570 580 590 600
CQPVLPSPAC QLVLHQILKG GSGTYCLNVS LADTNSLAVV STQLIMPGQE AGLGQVPLIV
                610 620 630 640 650 660 661
GILLVLMAVV LASLIYRRRL MKQDFSVPQL PHSSSHWLRL PRIFCSCPIG ENSPLLSGQQ V
hLAMP-hgp100 (sequence of human LAMP lumenal domain and TM/cyt tail is
underlined; sequence of full length of human gp100 is italicized.)
(SEQ ID NO: 13)
                10 20 30 40 50 60
MAAPGSARRP LLLLLLLLLL GLMHCASAAM FMVKNGNGTA CIMANFSAAF SVNYDTKSGP
                70 80 90 100 110 120
KNMTFDLPSD ATVVLNRSSC GKENTSDPSL VIAFGRGHTL TLNFTRNATR YSVQLMSFVY
              130 140 150 160 170 180
NLSDTHLFPN ASSKEIKTVE SITDIRADID KKYRCVSGTQ VHMNNVTVTL HDATIQAYLS
              190 200 210 220 230 240
NSSFSRGETR CEQDRPSPTT APPAPPSPSP SPVPKSPSVD KYNVSGTNGT CLLASMGLQL
              250 260 270 280 290 300
NLTYERKDNT TVTRLLNINP NKTSASGSCG AHLVTLELHS EGTTVLLFQF GMNASSSRFF
              310 320 330 340 350 360
LQGIQLNTIL PDARDPAFKA ANGSLRALQA TVGNSYKCNA EEHVRVTKAF SVNIFKVWVQ
              370 380 390 400 410 420
AFKVEGGQFG SVEECLLDEN SMDLVLKRCL LHLAVIGALL AVGATKVPRN QDWLGVSRQL
              430 440 450 460 470 480
RTKAWNRQLY PEWTEAQRLD CWRGGQVSLK VSNDGPTLIG ANASFSIALN FPGSQKVLPD
              490 500 510 520 530 540
GQVIWVNNTI INGSQVWGGQ PVYPQETDDA CIFPDGGPCP SGSWSQKRSF VYVWKTWGQY
              550 560 570 580 590 600
WQVLGGPVSG LSIGTGRAML GTHTMEVTVY HRRGSRSYVP LAHSSSAFTI TDQVPFSVSV
              610 620 630 640 650 660
SQLRALDGGN KHFLRNQPLT FALQLHDPSG YLAEADLSYT WDFGDSSGTL ISRALVVTHT
              670 680 690 700 710 720
YLEPGPVTAQ VVLQAAIPLT SCGSSPVPGT TDGHRPTAEA PNTTAGQVPT TEVVGTTPGQ
              730 740 750 760 770 780
APTAEPSGTT SVQVPTTEVI STAPVQMPTA ESTGMTPEKV PVSEVMGTTL AEMSTPEATG
              790 800 810 820 830 840
MTPAEVSIVV LSGTTAAQVT TTEWVETTAR ELPIPEPEGP DASSIMSTES ITGSLGPLLD
              850 860 870 880 890 900
GTATLRLVKR QVPLDCVLYR YGSFSVTLDI VQGIESAEIL QAVPSGEGDA FELTVSCQGG
              910 920 930 940 950 960
LPKEACMEIS SPGCQPPAQR LCQPVLPSPA CQLVLHQILK GGSGTYCLNV SLADTNSLAV
              970 980 990 1000 1010 1020
VSTQLIMPGQ EAGLGQVPLI VGILLVLMAV VLASLIYRRR LMKQDFSVPQ LPHSSSHWLR
            1030 1040 1050 1060 1070
LPRIFCSCPI GENSPLLSGQ QVMLIPIAVG GALAGLVLIV LIAYLVGRKR SHAGYQTI
D.D.-hpg100 (D.D. sequence is underlined; sequence of human gp100 minus
signal sequence and transmembrane domain is italicized.)
(SEQ ID NO: 14)
10 20 30 40 50 60
MGVQVETISP GDGRTFPKRG QTCVVHYTGM LEDGKKFDSS RDRNKPFKFM LGKQEVIRGW
                70 80 90 100 110 120
EEGVAQMSVG QRAKLTISPD YAYGATGHPG IIPPHATLVF DVELLKPEKV PRNQDWLGVS
              130 140 150 160 170 180
RQLRTKAWNR QLYPEWTEAQ RLDCWRGGQV SLKVSNDGPT LIGANASFSI ALNFPGSQKV
              190 200 210 220 230 240
LPDGQVIWVN NTIINGSQVW GGQPVYPQET DDACIFPDGG PCPSGSWSQK RSFVYVWKTW
              250 260 270 280 290 300
GQYWQVLGGP VSGLSIGTGR AMLGTHTMEV TVYHRRGSRS YVPLAHSSSA FTITDQVPFS
              310 320 330 340 350 360
VSVSQLRALD GGNKHFLRNQ PLTFALQLHD PSGYLAEADL SYTWDFGDSS GTLISRALVV
              370 380 390 400 410 420
THTYLEPGPV TAQVVLQAAI PLTSCGSSPV PGTTDGHRPT AEAPNTTAGQ VPTTEVVGTT
              430 440 450 460 470 480
PGQAPTAEPS GTTSVQVPTT EVISTAPVQM PTAESTGMTP EKVPVSEVMG TTLAEMSTPE
              490 500 510 520 530 540
ATGMTPAEVS IVVLSGTTAA QVTTTEWVET TARELPIPEP EGPDASSIMS TESITGSLGP
              550 560 570 580 590 600
LLDGTATLRL VRRQVPLDCV LYRYGSFSVT LDIVQGIESA EILQAVPSGE GDAFELTVSC
              610 620 630 640 650 660
QGGLPKEACM EISSPGCQPP AQRLCQPVLP SPACQLVLHQ ILKGGSGTYC LNVSLADTNS
              670
LAVVSTQLIM PGQEAG
s.s.-hgp100 (signal sequence of human gp100 is underlined; sequence of
human gp100 minus transmembrane domain is italicized.)
(SEQ ID NO: 15)
                10 20 30 40 50 60
MDLVLKRCLL HLAVIGALLA VGATKVPRNQ DWLGVSRQLR TKAWNRQLYP EWTEAQRLDC
                70 80 90 100 110 120
WRGGQVSLKV SNDGPTLIGA NASFSIALNF PGSQKVLPDG QVIWVNNTII NGSQVWGGQP
              130 140 150 160 170 180
VYPQETDDAC IFPDGGPCPS GSWSQKRSFV YVWKTWGQYW QVLGGPVSGL SIGTGRAMLG
              190 200 210 220 230 240
THTMEVTVYH RRGSRSYVPL AHSSSAFTIT DQVPFSVSVS QLRALDGGNK HFLRNQPLTF
              250 260 270 280 290 300
ALQLHDPSGY LAEADLSYTW DFGDSSGTLI SRALVVTHTY LEPGPVTAQV VLQAAIPLTS
              310 320 330 340 350 360
CGSSPVPGTT DGHRPTAEAP NTTAGQVPTT EVVGTTPGQA PTAEPSGTTS VQVPTTEVIS
              370 380 390 400 410 420
TAPVQMPTAE STGMTPEKVP VSEVMGTTLA EMSTPEATGM TPAEVSIVVL SGTTAAQVTT
              430 440 450 460 470 480
TEWVETTARE LPIPEPEGPD ASSIMSTESI TGSLGPLLDG TATLRLVRRQ VPLDCVLYRY
              490 500 510 520 530 540
GSFSVTLDIV QGIESAEILQ AVPSGEGDAF ELTVSCQGGL PKEACMEISS PGCQPPAQRL
              550 560 570 580 590
CQPVLPSPAC QLVLHQILKG GSGTYCLNVS LADTNSLAVV STQLIMPGQE AG
```

### Example 6

### Preparation of Dendritic Cell Vaccine Based on the Polycistronic Constructs

Peripheral blood mononuclear cells (PBMCs) from healthy donors were cultured in at 37° C. in a 5% CO₂ incubator for 2 hours. The adherent cells were then stimulated by 800 IU/ml GM-CSF and 500 IU/ml IL-4 in AIM-V media for 6 days to obtain immature dendritic cells (iDC). Target mRNA modified by domains was transcribed from constructs or synthesized in vitro. Then the mRNA was transfected in to iDC above to obtain mRNA loaded iDC. The mRNA loaded iDC was cultured for overnight with 800 IU/ml GM-CSF, 500 IU/ml IL-4, 160 ng/ml IL6, 5 ng/ml TNF-α, 5 ng/ml IL-1β and 1 μg/ml PGE2 to obtain mRNA loaded mature dendritic cells (mDC). The mRNA loaded mDC was harvested and then used as DC vaccine.

### Example 7

### Nanoparticle Delivery System

LNP formulations were generated in accordance with a slightly modified procedure as described by Chen et al. (2016) J. Control. Release, 235, 236-244. Lipids were dissolved in ethanol at molar ratios of 50:10:38.5:1.5 (ionizable lipid: DSPC: cholesterol: PEG-lipid). To the lipid mixture was added a 50 mM citrate buffer (pH 4.0) containing mRNA at a ratio of 3:1 (aqueous:ethanol) using a microfluidic mixer (Precision Nanosystems, Vancouver, BC). The resultant mixtures were dialyzed against PBS (pH 7.4) for at least 24 hours followed by concentration using Amicon Ultra Centrifugal Filters (EMD Millipore, Billerica, MA). The concentrated lipid nanoparticle solutions were passed through a 0.22-mm filter and stored at 4° C. prior to use. All formulations were tested for particle size, RNA encapsulation, and endotoxin to ensure that the lipid nanoparticles were between 80 and 100 nm, with greater than 90% encapsulation and <1 EU/ml of endotoxin.

Nanoparticle delivery system: The nanoparticle is made of polymer solution A, B, C and mRNA. First, 1 μg mRNA solution was added to 3 polymer solution A and 3 μl polymer solution B. Mixed well and incubated for 20 min at room temperature. Second, 2 μl polymer solution C was added, followed by the addition of NaOAc buffer up to 10 μl. Mixed well and incubated for another 20 min to obtain the final polyplex solution (nanoparticles). 10 μl polyplex solution was used for 1×10⁵ cells in one well of a 96 well plate.

Expression of nanoparticles delivered CMV-pp65 mRNA in DC. DC was prepared as described above. Add 10 μl polyplex solution above into 1×10⁵ cells in one well of a 96 well plate. DCs were cultured in 37° C. 5% CO₂ incubator and harvested at 6 h, 12 h and 24 h. Duplicated wells were set for each condition. The expression of CMV-pp65 in DC was measured by flow cytometry.

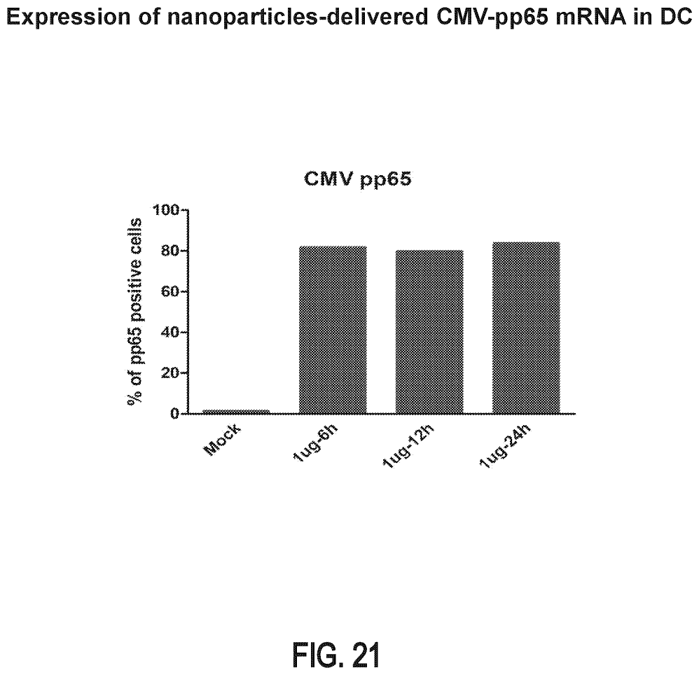

Results: CMV-pp65 mRNA was delivered successfully by nanoparticle delivery system and expressed efficiently in DC. Data showed that the percentage of CMV-pp65 positive cells was up to around 80% during the measured time (6 h, 12 h and 24 h). It indicated the target mRNA was successfully transfected in to DC by nanoparticle delivery. And the high CMV-pp65 expression efficiency proved that the target mRNA delivered by nanoparticles expressed well. (See, FIG. 21)

Expression of nanoparticles delivered CMV-pp65 mRNA in DC. CMV-pp65 mRNA or mock control was transfected into DC by nanoparticles delivery system, in a concentration of 1 ug mRNA/1×105 cells. DCs were cultured in 37° C. 5% CO2 incubator and harvested at 6 h, 12 h and 24 h. Duplicated wells were set for each condition. The expression of CMV-pp65 in DC was measured by flow cytometry. (See, FIG. 21)

### Example 8

### MHC-I/Peptide Antigen Presentation Enhanced by DD-Modification

### Materials and Methods:

In Vitro Culture of BM-Derived DCs (BMDCs): The bone marrow cells from mice were cultured in tissue-culture-treated plates in complete medium (RPMI-1640 supplemented with 10% heat-inactivated FBS [BenchMark], L-glutamine [Gibco], penicillin/streptomycin [Gibco], Gentamicin [Gibco], Sodium pyruvate [SIGMA], 2-mercaptoethanol [Gibco]). Subsequently, GM-CSF (Peprotech) and IL-4 (Peprotech) were added into the medium to a final concentration of 20 ng/mL and 5 ng/mL, respectively. The cells were cultured at 37° C. in an incubator containing 5% CO2. Half of the medium was removed every two days and added fresh warmed medium supplemented with GM-CSF (2×, 40 ng/ml) and IL-4 (2×, 10 ng/ml). On day 6, lipopolysaccharide (100 ng/ml) was added for an additional incubation for 24 h to induce DC maturation. On day 7, all cells harvested by washing with PBS were pooled.

Flow Cytometric Analysis of DC Phenotypes: The cells were washed with PBS, and divided into several fractions of 5×10⁵ cells/100 μl. FITC-labeled anti-CD11c and anti-CD14, PE-labeled anti-CD80, anti-CD83, anti-CD86, anti-H2kb, anti-IA/IE and anti-CCR7 (All from Biolegend) were added into the suspension and incubated for 20 min at 4° C. in the dark. The cells were washed with PBS twice and analyzed by flow cytometry. Fluorescence-labeled IgG isotypes were used as the control.

Transfection: A total of 2-10×10⁶ cells was suspended in 100 μl Mouse Dendritic Nucleofector Solution (Lonza) and transferred into a sterile electroporation cuvette (Lonza). Different plasmids (constructed with p43 expression vector) were added and then cells were electroporated (Nucleofector Program AN-001) by Nucleofector II Device (Lonza), and the ratio of BMDCs to plasmids was 1×10⁶ cells: 0.5 μg. To test transfection efficacy, BMDCs were transfected with pmaxGFP in parallel.

Flow Cytometric Analysis of the Expression of SIINFEKL/H2-Kb Complexes: The transfected BMDCs were washed with PBS, and then PE-labeled 25D 1.16 Ab (Biolegend) were added into the suspension and incubated for 20 min at 4° C. in the dark. The cells were washed with PBS twice and analyzed by flow cytometry.

### Results:

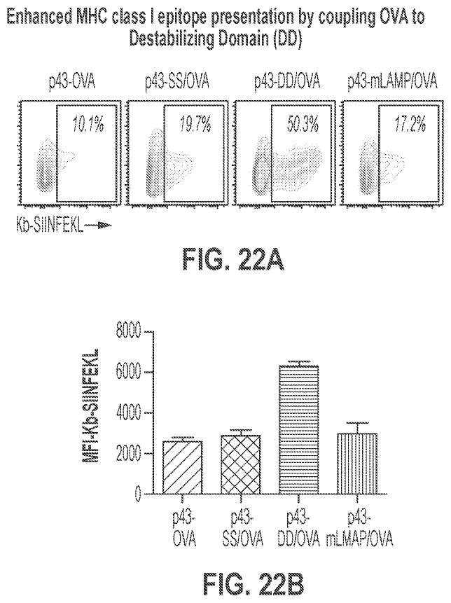

The commonly used antigen chicken ovalbumin (OVA) was cloned into the expression vector p43 as a control. The OVA antigen was modified by being linked to the SS, DD, or mouse LAMP1-domain. These modified OVA antigens were cloned separately into the p43 expression vector. The DNA plasmids were transfected into mature mouse DCs by electroporation. At different time points, DC cell surface expression of MHC-1/OVA peptide complexes was measured by PE-labeled 25D 1.16 Ab. PE-labeled 25D1.16 Ab directly binds to Kb-SIINFEKL mouse MHC-1/OVA peptide. As shown in FIG. 22A, the surface staining revealed that DCs transfected with DD/OVA had the most MHC-1/OVA peptide positive cells (50.3%). Furthermore, compared to p43-OVA control and other modification, DD/OVA had the highest cell surface expression of MHC-1/OVA peptide, more than double the expression level of the unmodified p43-OVA (FIG. 22B). These results demonstrated that DD modification of OVA resulted in dramatically enhanced MHC-I/OVA peptide antigen presentation.

### Example 9

Comparison of DD-Modification with Other Modifications on MHC-I/OVA Peptide Antigen Presentation

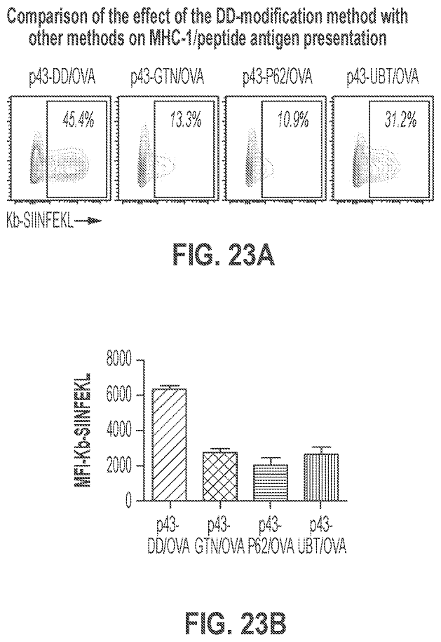

Modification with different molecular structures has been shown to enhance MHC-I/peptide antigen presentation. These included the selective autophagy receptor SQSTM1/p62 (Andersen A. N. et al., Front Immunol. 2016 May 10; 7:167), 7-tubulin (GTN) (Hung C. F. et al., Cancer Res. 2003 May 15;63(10):2393-8), and ubiquitin (UBT) (Hosoi A. et al., Biochem Biophys Res Commun. 2008 Jun. 27; 371(2):242-6). OVA was modified with the published method and cloned into the p43 expression vectors. Mature mouse DCs were transfected with the different plasmids and analyzed for surface MHC-I/OVA peptide expression. As shown in FIG. 23A, DD/OVA modification resulted in the most positive cells after transfection. Furthermore, DD/OVA modification had the highest cell surface expression of Kb-SIIFEKL MHC-1/OVA peptide (FIG. 23B). These results demonstrated that DD modification of OVA is superior to the other three known modifications in enhancing MHC-I antigen presentation.

### Example 10

### In Vivo Anti-Tumor Efficacy of DD-OVA

### Materials and Methods:

Mice, Cell Lines, and Tumor Models: C57BL/6 mice were from the Jackson Laboratory, and bred in a specific pathogen-free barrier facility and used at 6-12 weeks of age. All studies were approved by the Duke University Animal Care and Use Committee. A B16/F10 melanoma tumor cell line expressing membrane-bound OVA (B16/F10/mOVA) was kindly provided by Dr. Thomas F. Tedder (Univ. Duke, Durham, NC). It was produced using an expression plasmid (pIRES2-EGFP) containing cDNA encoding full-length OVA protein linked to the transmembrane region of H-2Db. Cells expressing GFP at high levels were selected by multiple rounds of fluorescence-based cell sorting. Cells were passaged minimally and maintained in complete RPMI-1640 containing 10% FBS, 200 mg/ml penicillin, and 200 U/ml streptomycin. To maintain OVA expression, B16/F10/mOVA cell cultures contained G418 (400 μg/ml). A total of 5×10⁴ B16/F10/mOVA tumor cells in 100 μl PBS were s.c. inoculated into 6-to-12-week-old C57BL/6 mice. Advanced tumors were established on day 5 or 6, and then vaccination of mice was initiated. 1×10⁶ electroporated DCs (resuspended in 100 μl PBS) were injected i.p. on day 7 and 14. Tumor development was monitored daily. The mice were sacrificed on day 30.

### Results:

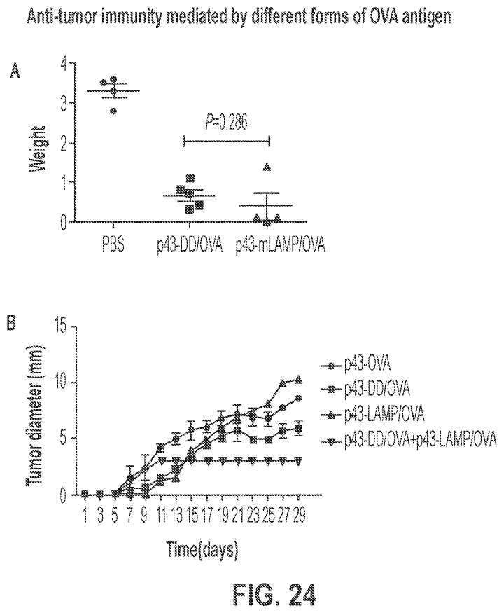

An in vivo anti-tumor efficacy assay was performed using DCs transfected with different forms of OVA antigen. As shown in FIG. 24A, DCs transfected with DD/OVA and mLAMP1/OVA plasmids mediated significant antitumor responses. Tumor weight from these two treatment groups was dramatically lower than the PBS treated control group. Furthermore, combined therapy with mixed DCs cells transfected with DD/OVA or mLAMP/OVA had a superior antitumor effect than DCs transfected with a single form of OVA antigen (FIG. 24B). These results suggest synergistic antitumor effect when combining MHC-I and MHC-II antigen modification methods by DD and LAMP1.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

1-41. (canceled)

42. A nucleic acid vector for expressing a target antigen for eliciting an enhanced antigen-specific T cell response, the vector encoding a fusion polypeptide comprising the target antigen and a destabilization domain (D.D.), wherein the D.D. is a destabilizing mutant of human FKBP12.

43. The nucleic acid vector of claim 42, wherein the fusion polypeptide further comprises a LAMP domain.

44. The nucleic acid vector of claim 42, wherein the target antigen is derived from a pathogen, a human self-protein, a tumor antigen, or a malignant neoplasm.

45. The nucleic acid vector of claim 44, wherein the target antigen is cytomegalovirus (CMV) pp65.

46.-70. (canceled)

71. The nucleic acid vector of claim 42, wherein the fusion polypeptide further comprises a signal sequence (s.s.).

72. The nucleic acid vector of claim 42, wherein the nucleic acid vector comprises a DNA construct, RNA construct, or any combination thereof.

73. The nucleic acid vector of claim 44, wherein the tumor antigen is a tumor specific antigen, a tumor associated antigen or a neoantigen.

74. The nucleic acid vector of claim 44, wherein the target antigen is a tumor antigen selected from the group consisting of 5T4, AIM2, AKAP4 2, Art-4, Aura A1 (AURKA), Aura B1 (AURKB), BAGE, BCAN, B-cyclin, BSG, CCND1, CD133, CDC45L, CDCA1 (TTK), CEA, CHI3L2 (Chitinase 3-like 2), CSPG4, EpCAM 4, Epha2, EPHX1, Ezh2, FABP7, Fos11 (Fra-1), GAGE, Galt-3, G250 (CA9), gBK, glast, GnT-V, gp100 (human gp100), HB-EGF, HER2, HNPRL, HO-1, hTERT, IGF2BP3, IL13-Ra2, IMP-3, IQGAP1, ITGAV, KIF1C, KIF20A, KIF21B, KIFC3, KK-LC-1, LAGE-1, Lck, LRRC8A, MAGE-1 (MAGEA1), MAGE-2 (MAGEA2B), MAGE-3, MAGE-4, MAGE-6, MAGE-10, MAGE-12, MAGE-C1 (CT7), MAGE-C2, MAGE-C3, Mart-1, MELK, MRP3, MUC1, NAPSA, NLGN4X, Nrcam, NY-ESO-1 (CTAGIB), NY-SAR-35, OFA/iLRP, PCNA, PIK3R1, Prame, PRKDC, PTH-rP, PTPRZ1, PTTG1 2, PRKDC, RAN, RGS1, RGS5, RHAMM (RHAMM-3R), RPL19, Sart-1, Sart-2, Sart-3, SEC61G, SGT-1, SOX2, Sox10, Sox11, SP17, SPANX-B, SQSTM1, SSX-2, STAT1, STAT3, Survivin, TARA, TNC, Trag-3, TRP-1, TRP2, Tyrosinase, URLC10 (LY6K), Ube2V, WT1, XAGE-lb (GAGED2a), YKL-40 (CHI3L1), ACRBP, SCP-1, SSX-1, SSX-4, NY-TLU-57, CAIX, Brachyury, NY-BR-1, ErbB, Mesothelin, EGFRvIII, IL-13Ra2, MSLN, GPC3, FR, PSMA, GD2, L1-CAM, VEGFR1, VEGFR2, KOC1, OFA, SL-701, Mutant P53, DEPDCI, MPHOSPHI, ONT-10, GD2L, GD3L, TF, PAP, BRCA1 DLC1, XPO1, HIF1A, ADAM2, CALR3, SAGE1, SCP-1, ppMAPkkk, WHSC, Mutant Ras, COX1, COX2, FOXP3, IDO1, ID02, TDO, PDL1, PDL2, and PGE2.

75. The nucleic acid vector of claim 74, wherein the tumor antigen is a human gp100 tumor associated antigen.

76. The nucleic acid vector of claim 44, wherein the target antigen derived from a pathogen is a viral pathogen.

77. The nucleic acid vector of claim 76, wherein the viral pathogen is selected from the group consisting of influenza virus, human papillomavirus (HPV), hepatitis B virus (HBV), hepatitis C virus (HCV), Epstein-Barr virus (EBV), dengue virus, and human immunodeficiency virus (HIV).

78. The nucleic acid vector of claim 42, wherein the destabilizing mutant of human FKBP12 comprises FKBP12 mutations selected from the group consisting of F15S, V24A, L30P, E60G, M66T, R71G, D100N, E102G, K105I, E107G, L106P, and any mutations or combinations thereof.

79. The nucleic acid vector of claim 78, wherein the destabilizing mutant of FKBP12 comprises the L106P mutation.

80. A vaccine composition comprising the nucleic acid vector of claim 42.

81. The vaccine composition of claim 80, wherein the vaccine composition further comprises:
(a) a nucleic acid vector encoding a fusion polypeptide comprising the target antigen and a lysosome-associated membrane protein (LAMP) domain; and/or
(b) a nucleic acid vector encoding a fusion polypeptide comprising the target antigen and a signal sequence (s.s.).

82. The vaccine composition of claim 80, wherein the destabilizing mutant of human FKBP12 comprises FKBP12 mutations selected from the group consisting of F15S, V24A, L30P, E60G, M66T, R71G, D100N, E102G, K105I, E107G, L106P, and any mutations or combinations thereof.

83. A method of modulating an immune response in a subject comprising administering the vaccine composition of claim 80.

84. A method of modulating an immune response in a subject comprising administering the vaccine composition of claim 81.

85. A method of eliciting an enhanced antigen-specific vaccination in a subject comprising administering the vaccine composition of claim 80.

86. A method of eliciting an enhanced antigen-specific vaccination in a subject comprising administering the vaccine composition of claim 81.


SEQUENCE LISTING (15 sequences)

SEQ ID NO: 1 - protein, 107 aa, Homo sapiens
GVQVETISPGDGRTFPKRGQTCVVHYTGMLGDGKKVDSSRDRNKPFKFMLGKQEVIRGWEEGVAQMSVGQGAKLTISPDYAYGATGHPGIIPPHATLVFDVELLELE

SEQ ID NO: 2 - protein, 417 aa, Homo sapiens
MAAPGSARRPLLLLLLLLLLGLMHCASAAMFMVKNGNGTACIMANFSAAFSVNYDTKSGPKNMTFDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSMLIPIAVGGALAGLVLIVLIAYLVGRKRSHAGYQTI

SEQ ID NO: 3 - protein, 24 aa, Homo sapiens
MGKRSAGSIMWLASLAVVIACAGA

SEQ ID NO: 4 - protein, 575 aa, Encephalomyocarditis virus
CGGGATCAATTCCGCCCCCCCCCTAACGTTACTGGCCGAAGCCGCTTGGAATAAGGCCGGTGTGCGTTTGTCTATATGTTATTTTCCACCATATTGCCGTCTTTTGGCAATGTGAGGGCCCGGAAACCTGGCCCTGTCTTCTTGACGAGCATTCCTAGGGGTCTTTCCCCTCTCGCCAAAGGAATGCAAGGTCTGTTGAATGTCGTGAAGGAAGCAGTTCCTCTGGAAGCTTCTTGAAGACAAACAACGTCTGTAGCGACCCTTTGCAGGCAGCGGAACCCCCCACCTGGCGACAGGTGCCTCTGCGGCCAAAAGCCACGTGTATAAGATACACCTGCAAAGGCGGCACAACCCCAGTGCCACGTTGTGAGTTGGATAGTTGTGGAAAGAGTCAAATGGCTCTCCTCAAGCGTATTCAACAAGGGGCTGAAGGATGCCCAGAAGGTACCCCATTGTATGGGATCTGATCTGGGGCCTCGGTGCACATGCTTTACATGTGTTTAGTCGAGGTTAAAAAAACGTCTAGGCCCCCCGAACCACGGGGACGTGGTTTTCCTTTGAAAAACACGATAATA

SEQ ID NO: 5 - protein, 21 aa, Thoseaasigna virus
GSGEGRGSLLTCGDVEENPGP

SEQ ID NO: 6 - protein, 22 aa, Porcine tesochovirus-1
GSGATNFSLLKQAGDVEENPGP

SEQ ID NO: 7 - protein, 23 aa, Equine rhinitis A virus
GSGQCTNYALLKLAGDVESNPGP

SEQ ID NO: 8 - protein, 25 aa, Foot and mouth disease virus
GSGVKQTLNFDLLKLAGDVESNPGP

SEQ ID NO: 9 - protein, 796 aa, synthetic construct
  REGION 1..796: note = Synthesized sequence mLAMP-OVA
MAAPGARRPLLLLLLAGLAHGASALFEVKNNGTTCIMASFSASFLTTYETANGSQIVNISLPASAEVLKNGSSCGKENVSDPSLTITFGRGYLLTLNFTKNTTRYSVQHMYFTYNLSDTEHFPNAISKEIYTMDSTTDIKADINKAYRCVSDIRVYMKNVTVVLRDATIQAYLSSGNFSKEETHCTQDGPSPTTGPPSPSPPLVPTNPTVSKYNVTGNNGTCLLASMALQLNITYLKKDNKTVTRAFNISPNDTSSGSCGINLVTLKVENKNRALELQFGMNASSSLFFLQGVRLNMTLPDALVPTFSISNHSLKALQATVGNSYKCNTEEHIFVSKMLSLNVFSVQVQAFKVDSDRFGSVEECVQDGNNVDMGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPTSMLIPIAVGGALAGLVLIVLIAYLVGRKRSHAGYQTI

SEQ ID NO: 10 - protein, 495 aa, synthetic construct
  REGION 1..495: note = Synthesized sequence D.D.-OVA
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPEFGVQVETISPGDGRTFPKRGQTCVVHYTGMLGDGKKVDSSRDRNKPFKFMLGKQEVIRGWEEGVAQMSVGQGAKLTISPDYAYGATGHPGIIPPHATLVFDVELLELE

SEQ ID NO: 11 - protein, 412 aa, synthetic construct
  REGION 1..412: note = Synthesized Sequence s.s.-OVA
MGKRSAGSIMWLASLAVVIACAGACTMGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 12 - protein, 661 aa, Homo sapiens
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 13 - protein, 1078 aa, synthetic construct
  REGION 1..1078: note = Synthesized Sequence hLAMP-hgp100
MAAPGSARRPLLLLLLLLLLGLMHCASAAMFMVKNGNGTACIMANFSAAFSVNYDTKSGPKNMTFDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSMDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQVMLIPIAVGGALAGLVLIVLIAYLVGRKRSHAGYQTI

SEQ ID NO: 14 - protein, 676 aa, synthetic construct
  REGION 1..676: note = Synthesized Sequence D.D.-hgp100
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKPEKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAG

SEQ ID NO: 15 - protein, 592 aa, synthetic construct
  REGION 1..592: note = Synthesized Sequence s.s.-hgp100
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAG